(19) United States
(12) Patent Application Publication
(10) Pub. No.: US 2013/0273580 A1
(43) Pub. Date: Oct. 17, 2013

(54) ELISA ASSAY KIT, ELISA FOR DETERMINING DESMOSINE LEVELS FROM URINE SAMPLES AND DIAGNOSTIC URINE ASSAYS FOR ANEURYSMS

(71) Applicants: Barry C. Starcher, Bullard, TX (US); Marina A. Durward, Stillwater, MN (US); Laurie Lynch, Eden Prairie, MN (US); Matthew F. Ogle, Fitchburg, WI (US)

(72) Inventors: Barry C. Starcher, Bullard, TX (US); Marina A. Durward, Stillwater, MN (US); Laurie Lynch, Eden Prairie, MN (US); Matthew F. Ogle, Fitchburg, WI (US)

(21) Appl. No.: 13/448,734

(22) Filed: Apr. 17, 2012

(51) Int. Cl. G01N 33/53; G01N 21/76

(52) U.S. Cl. 435/7.93

(57) ABSTRACT

Improved ELISA assay formats are described that provide for effective measurement of desmosine, an elastin degradation product, in urine samples. The urine samples can be effectively introduced into the assay with little or no sample preparation. The competitive ELISA assay based on high titer polyclonal antibodies is suitable for commercial application. Desirable antibodies can be generated using desmosine bound to a protein or the like using a protein crosslinking agent. The desmosine assay are found to be useful for the diagnosis of aortic aneurysms in which desmosine levels of patients with aortic aneurysms have been found to be significantly elevated relative to a control group.

### FIELD OF THE INVENTION

The invention relates to assays for desmosine, an elastin breakdown product, from urine samples. The invention further relates to kits for performing an assay, generally an ELISA assay, for desmosine as well as to methods for performing the desmosine assay. Desmosine levels from urine are described as being diagnostic for aneurysm risk.

### BACKGROUND OF THE INVENTION

Fibrillar proteins elastin and collagen (types I and III) are the principal structural proteins of the aorta and other arteries, which impart both strength and resilience to the vessel wall. Elastin in particular endows vascular tissue with the ability to extend and recoil repetitively. Elastin is primarily composed of the amino acids glycine, valine, alanine, and proline. It is a specialized protein with an irregular or random coil conformation. Elastin is made by linking many soluble tropoelastin protein molecules, in a reaction catalyzed by lysyl oxidase, to make a massive insoluble, durable cross-linked array. Due to its insolubility and extremely long biological half-life, elastin is generally perceived to be resistant to degradation. However, degradation of elastin can be associated with a disease state.

Desmosine and isodesmosine are integral components of elastin, formed from four side chains of lysine of soluble tropoelastin protein and constituting cross linkages in the insoluble elastin. The structure of desmosine is shown in FIG. 1a, with a pyridinium core having four amino carboxyl side chains at 1, 3, 4, and 5 position of the pyridinium core. Isodesmosine is a structural isomer of desmosine, with four amino carboxyl side chains at 1, 2, 3, and 5 position of the pyridinium core, as shown in FIG. 1b. Desmosine and isodesmosine are amino acid derivatives that are specific to elastin. When elastin goes through degradation, desmosine and isodesmosine are released as the elastin degradation products. Additionally, desmosine and isodesmosine are not metabolized and are passed to urine for removal from the body.

Aneurysms are degenerative diseases characterized by destruction of arterial architecture and subsequent dilatation of the blood vessel that may eventually lead to fatal ruptures. Some common locations for aneurysms include the abdominal aorta (abdominal aortic aneurysm, AAA), thoracic aorta, and brain arteries. In addition, peripheral aneurysms of the leg, namely the popliteal and femoral arteries are prevalent locations of this vascular pathology. Aneurysms grow over a period of years and pose great risks to health. Aneurysms have the potential to dissect or rupture, causing massive bleeding, stroke, and hemorrhagic shock, which can be fatal in a large percentage of cases.

### SUMMARY OF THE INVENTION

In a further aspect, the invention pertains to a method for performing an Enzyme-Linked Immunosorbent Assay (ELISA) for quantitative determination of desmosine levels in an non-hydrolyzed urine sample of a mammal, generally a human patient, a pet animal or a farm animal, the method comprising:

incubating the non-hydrolyzed urine sample with an anti-desmosine antibody a well with a desmosine-capture conjugate to form a competitive combination for a first incubation time of no more than about 6 hours, wherein the antibody has appropriate affinity for both the soluble desmosine and for the bound desmosine;

washing the well to remove anti-desmosine antibody not bound to the desmosine-capture conjugate;

adding an enzyme conjugated anti-antibody to the well to form a detection enabled sample well that is incubated for a second incubation time;

washing the well to remove unbound enzyme conjugated anti-antibody;

developing the detection enabled sample well by incubating an enzyme substrate with the detection enabled sample well for a third incubation time to form a detectable product;

detecting the amount of detectable product; and

estimating the amount of sample desmosine based on a comparison of the detected amount of detectable product with a standard curve. The ELISA assay results can be further used for diagnosing aneurysm. The diagnosis method can comprise,

detecting the level of desmosine in the urine sample using the ELISA; and

comparing the detected level of desmosine against a reference level of desmosine, wherein the reference level has been obtained through the ELISA measurement of the level of desmosine detected from urine sample of healthy individuals to determine if a patient should be flagged as a likely suffering from an aneurysm.

In another aspect, the invention pertains to a method for generating a standard curve for an Enzyme-Linked Immunosorbent Assay (ELISA) for quantitative determination of desmosine in a urine sample of a patient, the method comprising,

incubating an anti-desmosine antibody with a set of desmosine standard solutions each having a selected amount of desmosine spanning a desired range of desmosine concentrations in a separate wells having a selected amount of a desmosine-capture conjugate wherein desmosine-capture conjugate comprises a capture macromolecule, wherein the antibody has appropriate affinity for both the soluble desmosine and for the desmosine-capture conjugate and the well has an intermediate amount of desmosine-capture conjugate to provide a desired slope for a standard curve generated from the standard sample measurements over the range of concentrations corresponding to patient samples;

developing the incubated wells to illicit detectable signals from enzyme reaction products corresponding to the amount of desmosine in the standard; and

generating a standard curve based on the amount of signal measured as a function of desmosine concentrations in the standard samples.

In an additional aspect, the invention pertains to an Enzyme-Linked Immunosorbent Assay (ELISA) kit for quantifying desmosine in urine comprising a high titer polyclonal anti-desmosine antibody raised with desmosine bound to a protein using a multifunctional protein crosslinking agent and a desmosine-capture conjugate comprising desmosine bound to a capture macromolecule through a linker molecule. Generally, the desmosine-capture conjugate is effective for capture in a prepared well to effectively segregate the desmosine-capture conjugate from soluble sample desmosine and wherein the desmosine-capture conjugate is effective to compete with un-bound desmosine for the anti-desmosine antibody and wherein the high titer polyclonal anti-desmosine antibody quantitatively captures un-bond desmosine from non-hydrolyzed urine with no more than a 6 hour incubation time.

### BRIEF DESCRIPTION OF THE DRAWINGS

FIG. 1a is a structural diagram of desmosine.

FIG. 1b is a structural diagram of isodesmosine.

FIG. 2 is a set of standard curves of desmosine standards using the ELISA assay of example 4.

FIG. 3 shows distribution of radioimmunoassay (RIA) results by group according to example 10.

FIG. 4 shows specificity versus sensitivity of the RIA assay of example 10.

FIG. 5 shows the comparison testing of RIA versus ELISA.

### DETAILED DESCRIPTION OF THE INVENTION

An efficient Enzyme-Linked Immunosorbent Assay (ELISA) for desmosine, an elastin degradation product, is presented for urine samples with a desired high degree of sensitivity and selectivity. Results are presented indicating the usefulness of the assay for the diagnosis of aneurysms. The assay is based on high titer polyclonal anti-desmosine antibodies. As demonstrated below, the anti-desmosine antibodies seem to cross react with isodesmosine. Thus, unless specifically indicated otherwise, references to desmosine measurement refer to potentially combined measurements of desmosine and isodesmosine based on the use of anti-desmosine antibodies. The polyclonal antibodies are effectively incorporated into a competitive assay for the measurement of desmosine levels in a urine sample. The antibodies are generated under conditions to provide reasonable competition between soluble sample desmosine and conjugated desmosine. Also, it has been found that surprisingly improved results are obtained if the sample desmosine, the desmosine antibody and the conjugated desmosine are all incubated together, in contrast with incubating the sample desmosine first separately with the anti-desmosine antibody. Furthermore, generally using the improved antibody and improved incubation format, the amount of capture desmosine can be adjusted to produce a standard curve with a greater slope to provide more reproducible measurements. Through the effective design of the assay, urine samples can be used with little or no preparation of the samples without intolerable matrix effects. However, the assay is sensitive to modest dilution of the urine in the assay to reduce the matrix effect without sufficiently without lowering the assay sensitivity. Furthermore, the effective use of assay design allows for low incubation times for more efficient assay performance.

The assay can further incorporate appropriate capture formats, such as wells associated with appropriately selected microtiter plates that in combination provide for effective capture of desmosine from a sample. In particular, the capture structures or wells can be formed using desmosine conjugates that are immobilized in the wells or other structure to provide for effective capture of unbound anti-desmosine antibodies. The amount of bound desmosine can be selected at an intermediate value to provide for better effectiveness of the assay, and the binding of desmosine can be selected to achieve appropriate competition for antibody binding relative to the free desmosine. An effective detection system is described to provide for detection at desired concentrations.

An ELISA assay is designed to result in the quantitative binding of an enzyme linked antibody to a fixed structure, such as a well of a microtiter plate, based on the amount of analyte introduced into the assay. A substrate is added to react with the bound enzyme to quantitatively produce a detectable product of the reaction of the substrate with the enzyme. The detectable product is then detected to produce a quantitative signal. In some embodiments described herein, the detection product is visibly detectable through a color change. Specifically, for some embodiments, the absorption of light at a selected frequency can be used to measure the amount of detectable product. In the competitive assays described herein, a greater measurement of the detection product corresponding to the presence of the enzyme is correlated with a smaller amount of desmosine in a sample.

Desmosine is a hapten, which means that desmosine does not effectively generate antibodies when injected into an animal without attachment of the desmosine to an antigenic molecule to form a conjugate to facilitate antibody production. The attachment process can be significant since the generation of antibodies recognizing the desmosine antigen with a good affinity and a high titer are highly desirable. In general, the conjugate comprises a protein or other macromolecule that is antigenic with respect to the animal that is used to generate that antibody. Suitable animals for generating the antibodies include, for example, rabbits, chicken and mice. For the anti-desmosine antibodies, an effective approach for the generation of high titer polyclonal antibodies using a keyhole limpet hemocyanin protein in the conjugate. High titer polyclonal antibodies can be effective generated with these conjugates. Desmosine conjugates are also used to compete for anti-desmosine antibodies in the assay, and the formation of these conjugates is also very significant.

Sample desmosine and a desmosine conjugate used for capture are mixed with an anti-desmosine antibody in the competitive format. But a complex interplay of factors affects this competition. The desmosine antibody is generated with a desmosine-conjugate, and since the desmosine is a relatively small molecule, the antigenic site can be significantly influenced by the presence of the antigenic macromolecule bound to the desmosine during generation of the antibodies. Thus, if proper consideration is not given to these factors, the competition between the sample desmosine and the desmosine capture conjugate can be biased in favor of one element, presumably the desmosine conjugate, and the assay sensitivity can decrease below desired levels.

It has been found that good assay sensitivity resulting from good competition for the anti-desmosine antibody if the antibody is generated with desmosine conjugated with a protein using non-specific protein crosslinking agent. A particularly useful and convenient crosslinking agent is glutaraldehyde, which has been used for a long time as a protein crosslinking agent. The crosslinking agent generally can attach a plurality of desmosine molecules to each protein molecule to generate a plurality of antigenic sites with the desmosine. Suitable proteins generally are antigenic in the animal used to generate the antibodies. High titer antibodies have been generated in this way. In contrast, the capture macromolecule-desmosine conjugate can be formed with a specific difunctional linker that provides a gentler bonding of the desmosine to a capture macromolecule. Thus, with a gentler bonding in the capture conjugate, the desmosine in the conjugate more closely resembles the antigenic sample desmosine, and the capture desmosine conjugate does not closely resemble the antigenic conjugate used to raise the antibodies. Through this improved selection of the desmosine conjugates and the corresponding generation of the antibodies, dramatically improved assay results can be obtained.

In the competitive assay, desmosine in the sample and desmosine capture conjugate compete for the anti-desmosine antibody. An effective and efficient competitive ELISA assay is described herein based on the high titer anti-desmosine antibodies along with an effective desmosine capture conjugate in a specifically designed assay format. Furthermore, for embodiments of particular interest, it has surprisingly been found to be very effective to perform the competition for the anti-desmosine antibody by combining the antidesmosine antibody with both the sample desmosine and the capture desmosine conjugate at the same time. Of course, this should not be interpreted as necessarily involving the precise simultaneous combination of these components but as involving the combination of these components for the substantial majority of the incubation time so that the elements can be combined sequentially if desired. In contrast, more traditional ELISA assays involve the initial incubation of the antibody and sample antigen separately for a substantial incubation time prior to combination with the capture desmosine conjugate. However, if the high titer anti-desmosine antibodies are used in the traditional ELISA format with initial incubation with the sample desmosine, poor assay results are obtained, which may be due to poor competition between the sample desmosine and the desmosine conjugate. Thus, it has been surprisingly discovered that significant improvements result if the anti-desmosine antibody is combined with both the sample desmosine and the capture desmosine for a first incubation period. It has further been discovered that the improved format and improved anti-desmosine antibodies provide for a surprisingly short incubation time with the sample desmosine of no more than about 6 hours.

For convenience, the container used to perform assay binding is referred to as a well without particular reference to the size or shape of the container or the association of the container with a plate comprising a plurality of wells, although commercial microtiter plates can be a convenient format for the wells. For incorporation into an automated system, a well can be a convenient container suitable for automated fluid dispensing and manipulation. The well can be used to bind the capture desmosine conjugate. For example, the desmosine capture conjugate can be bound to a microtiter well prior to performing the incubation with the anti-desmosine antibody. In additional or alternative embodiments, the desmosine capture conjugate can be immobilized in the well during and/or following the incubation period. For example, the capture conjugate can comprise a magnetic moiety, such as a magnetic bead, that can be used to bind the conjugate alone or bound to an anti-desmosine antibody to the well using a magnet or the like. After incubating the antibodies with the capture desmosine conjugate and immobilizing the capture desmosine conjugate in the order and according to the selected format, the well can be washed to remove antibodies bound to sample desmosine free in the solution.

The washed wells with anti-desmosine antibodies bound to immobilized desmosine can be used to quantify the amount of bound antibody. A larger amount of bound antibody indicates a smaller amount of desmosine in the sample and correspondingly, a smaller amount of bound antibody indicates a larger amount of desmosine in the sample. A detection antibody can be added to the wells to bind to the bound anti-desmosine antibody in the wells. The detection antibody can comprise a conjugate with an antibody specific for binding the anti-desmosine antibody conjugated with a detection enzyme, e.g., horseradish peroxidase. The detection antibody is incubated with the well for a detection incubation time. Any unbound detection antibody can be washed from the well after the end of the detection incubation time.

A substrate for the enzyme is incubated with the washed well with bound detection antibodies for an appropriate time to allow the substrate to react with the enzyme. embodiments of particular interest, the substrate reacts with the enzyme to form product compound with a specific color that can be detected with optical measurements. A desirable substrate for the horseradish peroxidase is tetramethyl benzidine, although other substrates can be used as described below. The detection substrate can be incubated with the bound enzyme for a development incubation time prior to measurement. A stop solution can be added optionally at the end of the development incubation time to halt further reaction of the substrate to allow time for the optical measurement without altering the quantification of the reaction. The color produced from the reaction of the substrate with the bound enzyme can be read at an appropriate light wavelength for the product, for example, using a plate reader or the like.

The quantification of the desmosine in a urine sample from a patient relies on the comparison of the measured product of the detection substrate against values of standard desmosine solutions plotted in a standard curve. The standard desmosine solutions can be formed with concentrations over an appropriate range of concentrations to be meaningful for the designed ELISA assay. To form the standard curve, the assay is performed using the desmosine standard samples. The assay measurements based on the standard samples are then plotted as a function of the desmosine concentrations in the standards to obtain the standard curve. Using the improved assay formats and improved anti-desmosine antibodies as described herein, the slope of the standard curve can be increased relative to previously explored assay, and this improved sensitivity can provide for more accurate and reproducible sample measurements. An optical measurement then from an assay on a patient's urine sample can then be used to determine a desmosine concentration based on the standard curve by reading a desmosine concentration for a particular optical measurement.

In some embodiments, the ELISA assay described herein can be performed on a patient's urine samples with little or no preparation for the urine samples. To reduce matrix effects, urine can be diluted, but a great dilution is not desirable from a sensitivity perspective. Also, a urine sample can be subjected to acid hydrolysis, which can be used to free any desmosine in complexes in the urine, but acid hydrolysis is not desirable from a processing perspective. Acid hydrolysis can be potentially a prohibitive expense and complication in the context of a commercial assay. The improved assay described herein can be performed in some embodiments without acid hydrolysis and with a level of dilution that sufficiently reduces matrix effects without decreasing sensitivity below desired levels. However, if the urine is not sufficiently diluted, good assay results are not obtainable. Thus, it has been discovered that a careful balance of dilution can achieve sufficient reduction of urine background matrix interference while obtaining sufficient sensitivity for aneurysm diagnosis. In contrast, Watanabe et al. concluded that a 24 acid hydrolysis was needed to quantify the desmosine levels. (Watanabe et al., Tokai J. Exp. Clin. Med. Vol. 14, pp. 347-356 (1989)). As described herein, correlation between measured urine desmosine levels and presence of an aneurysm does not require acid hydrolysis of the urine samples. Note that Watanabe used overnight incubations both for the first incubation time with the desmosine samples and the antibody and for the detection incubation time with the capture wells and the desmosine sample-antibody blends.

The reduced processing of the urine sample can be possible due to the improved assay format with the high titer polyclonal antibodies described herein, and with the ability to obtain desired levels of specificity over the urine background matrix effect even without significantly diluting the urine samples. The ability to use the urine samples with reduced or eliminated pre-processing of the urine samples reduces processing steps while obtaining a desirable assay with good specificity and sensitivity. Thus, the assay herein is well suited for adaptation to a commercial assay that can provide valuable diagnostic capabilities to a broad patient population.

The assays described herein can be adapted for commercial application for low cost and relatively high volume processing. Also, if desired, the assays can be adapted for incorporation into automated systems for performing the measurements. The relative speed, efficiency of processing, short incubation times, high sensitivity and/or high selectivity are consistent with performing the assays in a commercial setting.

Because the onset and progression of aneurysms are associated with enzymatic degradation of elastin, detection of a predetermined level of desmosine in patient blood or urine can be an indication of aneurysm. Desmosine is excreted form a patient through the urine, and urine levels of desmosine can thus be a potential diagnostic tool. Elevated urinary desmosine levels have been found to be diagnostic for chronic obstructive pulmonary disease. See Cocci et al., The International Journal of Biochemistry & Cell Biology Vol. 34, pp 594-604 (2002). Furthermore, increasing level of desmosine in patient urine sample relative to corresponding control values from healthy individuals can be correlated to the progression of aneurysm. Results presented herein provide validation of the use of a urine assay to provide useful diagnostic results for aneurysms with a reasonable number of false negative measurement and false positive measurements. Validation of aneurysm diagnosis using urine desmosine levels has been obtained with a radioimmunoassay on urine samples, which can be correlated with results using the improved ELISA assay described herein, as well as a measurement procedure validation study directly for the ELISA assay, so that the ELISA assay can be effectively used to obtain an effective diagnostic assay for aneurysms using the ELISA.

Previous results have examined the use of desmosine as a marker for aortic aneurysm. Osakabe et al. found poor differentiation based on desmosine levels between patients with an aortic aneurysm and control patients. (See, Oskabe et al., Biol. Pharm. Bull. Vol. 22(8), pp 854-857 (1999).) Oskabe et al. found an elevated desmosine level on average for the aortic aneurysm group relative to the control group, but the measured desmosine levels for a large fraction of the aneurysm group were at levels corresponding with the bulk of the control group. However, the results presented herein using the improved ELISA assay find good diagnostic utility of the desmosine marker to distinguish between aortic aneurysm and control patients. Thus, the ELISA desmosine assay has been validated as a desirable diagnostic tool for aneurysm diagnosis with appropriate levels of sensitivity.

### Anti-Desmosine Antibody and Desmosine Conjugate Preparation

Two desmosine conjugates are used in the context of the ELISA assay. A first conjugate is used to generate the antibodies, and a second conjugate is used to immobilize reference desmosine for the competitive assay. High titer and/or high affinity anti-desmosine antibodies have been produced through the attachment of the desmosine to a protein or other appropriate macromolecule. As noted above, desmosine is a hapten so the desmosine is conjugated with an antigenic macromolecule to generate antibodies. In particular, antibodies can be generated with a conjugate of keyhole limpet hemocyanin protein properly conjugated with the desmosine. However, other antigenic proteins or other macromolecules can be used in the conjugate as desired if the conjugate is formed in a way to maintain good antigenic access to the desmosine. Using the procedure described herein, desirable anti-desmosine antibodies have been generated with a high titer based on protein crosslinking agents to attach the desmosine to a protein to form an antigenic conjugate. Similarly, suitable capture conjugates have been formed with desmosine and a suitable protein, although other capture macromolecules can be suitable, that is useful for immobilizing desmosine with appropriate binding accessibility to the desmosine by the anti-desmosine antibodies. The capture conjugates can be formed with a more direct binding approach using a bifunctional linker under controlled binding conditions.

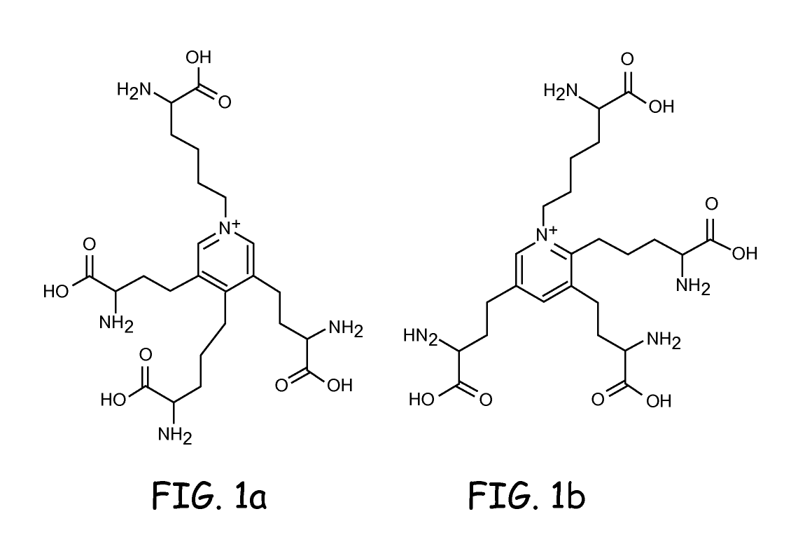

Desmosine is a specific polypeptide (or tetra (α-amino acid) only found in elastin. As shown in FIG. 1, desmosine has a pyridine ring with four chains extending from the ring and each chain terminated with an amino-carboxylic acid group. Desmosine is available from Elastin Products, although the desmosine used to develop the present assay was supplied by the present inventor based on the purification procedure described in Starcher et al., Preparative Biochemistry Vol. 5, p. 455 et seq. (1975), incorporated herein by reference.

To form the desmosine-protein conjugates, general protein crosslinking agents can be used, such as glutaraldehyde. This approach is desirable for the formation of antigenic desmosine conjugates, and surprisingly good antibodies for performing the ELISA assay have been generated using glutaraldehyde. Other similar protein crosslinking agents that crosslink between amine to amine groups include, for example, disuccinimidyl glutarate (DSG), disuccinimidyl subarate (DSS), bis(sulfosuccinimidyl) suberate (BS3), tris-succinimidyl aminotriacetate (TSAT), other NHS esters, and the like. As described in the examples below, a plurality of desmosine molecules can be bound to each protein molecule, and such bonding can be effective for the generation of high titer antibodies. Such aggressive bonding of the desmosine to a protein to form an antigenic macromolecule surprisingly results in the generation of effective anti-desmosine antibody. In general, a plurality of desmosine molecules can be bound to a single macromolecule. Crosslinking agents, such as glutaraldehyde, can further crosslink the surface of the protein with the surprising result that the resulting antibodies are effective in the competitive assay format described herein.

The desmosine immunogenic conjugates are injected into a suitable animal for the production of the antibodies based on reasonable immunological protocols. Suitable animals include, for example, rabbits, mice, chickens, or other mammals or birds. As noted above, keyhole limpet hemocyanin protein can be effectively used to generate antibodies in rabbits, although other antigenic proteins can be used as desired with suitable attachment. For example, rabbits or mice can be injected subcutaneously with the desmosine conjugate in an appropriate solution, such as buffered saline. Booster immunizations can be performed subcutaneously or intramuscularly every 2-3 weeks. Blood or other biological samples, such as eggs from chicken, can be collected at appropriate intervals, such as 1-2 weeks after each immunization injection.

Antibodies can be used using whole serum from an animal used to generate the antibodies. The results in the examples are based on the use of diluted whole serum. In additional or alternative embodiments, antibodies can be purified from the collected serum or other biological sample from the induced organism, such as eggs from a chicken. Column resin is available for the general purification of antibodies, e.g., IgG, obtained from the biological sample, and the resulting antibody fraction can be used in the ELISA assay. In addition, the antibodies can be purified using affinity purification in which the antigen, e.g., desmosine, is coupled to beads or the like for the performance of affinity chromatography, and affinity purified antibodies can be incorporated into the ELISA assay. Purification of the antibody can provide various tradeoffs with respect to assay performance, and in general a desired degree of antibody purification can be selected based on these tradeoffs. The titer of the antibodies can be evaluated using the binding to the antigen in the assay format.

Furthermore, a second desmosine conjugate, i.e., the capture desmosine conjugate, is used in the ELISA assay to fix desmosine to a well to compete with the sample desmosine. To form capture conjugates with desired exposure of the desmosine in the conjugate, more specific crosslinking agents can be desirable, and the crosslinking reactions can be controlled to correspondingly control the nature of the resulting conjugate. The capture desmosine conjugate comprises desmosine bound to a macromolecule that is selected to be suitable for binding to a fixed surface, e.g., a well wall, magnetic bead or the like, and suitable macromolecules can include proteins, such as ovalbumin or the like or linkages to bead surfaces as appropriate. Successful assay results are described below using ovalbumin as the binding macromolecule. The formation of the capture desmosine conjugate involves complimentary issues as the formation of the antigenic desmosine conjugate. Specifically, the anti-desmosine antibody should provide good competition between the sample desmosine and the desmosine in the capture conjugate, so that the antibody does not bind to an undesirable degree to one component of the competitive set.

Based on the high titer antibodies formed with a protein crosslinking agent, good competition in the competitive assay format has been obtained by binding of the desmosine to the fixation macromolecule using a specific difunctional linker that provides less aggressive type of bonding than the protein crosslinking agents used to form the conjugate to raise the antibodies. For capture conjugates, suitable linkers can comprise non-self-polymerizing, bifunctional linker molecules with two different functionalities. Good results are described herein based on 1-ethyl-3-(3-dimethylaminopropyl)-3-carbodiimide (EDC) coupling agent, although other bifunctional linkers with appropriate designs can be used. For example, other bifunctional linkers can include, for example, 1,5-difluoro-2,4-dinitrobenzene. Also, an excess amount of desmosine relative to the linker and macromolecule can be used to generate the fixation macromolecule to further having less extensive bonding of the desmosine so that the desmosine more closely resembles sample desmosine to the antibody and improved competition for the antibody can result.

### ELISA Assay Kit and Standards

Suitable ELISA assay components can comprise the high titer anti-desmosine antibody and the capture conjugate of desmosine and an immobilization macromolecule. These two components of assay kit are specifically engineered for the performance of an efficient ELISA assay in a competitive format, such as a commercial assay. These components are core components of the assay, but additional reagents can be appropriately selected for use together for the performance of the assay with the high performance parameters described herein. The improved performances achievable are described in the following section.

The particular concentrations for the assay components can depend on the particular assay format. For the assays described herein, urine samples are used without significant dilution, and corresponding dilutions can be referenced for this format. For the use of rabbit serum without further purification of the antibodies, the antibodies for use in the assay can have a dilution from about 1/1000 to about 1/25,000, in further embodiments from about 1/2000 to about 1/12,500 and in additional embodiments from about 1/3000 to about 1/10,000. In the assay format used in the examples, the antibody is diluted by approximately a factor of two within the assay well relative to the initial dilution of the antibody solution for addition to the assay container. The desired dilution can be somewhat dependent on the batch of serum with the antibody, and a person of ordinary skill in the art can adjust the dilution empirically if desired. A person of ordinary skill in the art will recognize that additional ranges within the explicit ranges of dilution above are contemplated and are within the present disclosure.

With respect to the desmosine capture conjugate, the desired concentrations can also depend on the particular format of the assay, the nature of the well or other capture format features and the like. As noted below, desired assay results can result from a balance of concentration of desmosine capture conjugate available during the antibody competition. The conjugate can be referenced relative to a weight of the conjugate, such that the dilution refers to the amount of liquid added with the conjugate. For the assay format described in the examples below using microtiter plates and a 100 microliter (μL) of coating solution, the conjugate can be added to the well to achieve from about 1 nanogram/well (ng/well) to about 100 ng/well, in further embodiments, from about 10 ng/well to about 80 ng/well and in additional embodiments from about 20 ng/well to about 60 ng/well. Of course, with different assay volumes and corresponding volumes of coating solution, the amounts of conjugates can be adjusted accordingly. Similarly, for other variations on the assay format, the amount of conjugate for a quantity of sample can be based on these values. A person of ordinary skill in the art will recognize that additional ranges of conjugate amounts within the explicit ranges above are contemplated and are within the present disclosure.

The particular volumes used in the assay can be adjusted according to the particular assay format. For distributions, of the anti-desmosine antibody or the desmosine conjugate can be, for example, in dried form, in a concentrated form or in a diluted form ready for use in the assay, as desired.

In addition to the anti-desmosine antibody and the desmosine conjugate, other significant components for the improved assay comprise, for example:

a container with a well;

enzyme-labeled anti-animal antibody (detection antibody);

enzyme substrate; and

optional enzyme stop solution.

A suitable container with a well can be a microtiter place with an array of wells or any other suitable container that is designed to capture the capture conjugate at an appropriate stage of the assay. The shape and volume of the well can be selected as desired, although for an ELISA assay, the volume of the well is generally relatively small. Working assay formats based on the use of wells in microtiter plates are described in the Examples below. For microtiter plates, the well should have binding sights to bind the macromolecule used to form the desmosine conjugates. As described above, in some embodiments, the conjugate can comprise ovalbumin such that the wells should have appropriate binding affinity for ovalbumin. Other macromolecules can be used for forming the conjugates, and the wells can be selected appropriately. The wells should have consistent binding of the desmosine conjugates and with a desired degree of desmosine binding based on the particular procedure.

To provide a desired degree of competition in the assay, the amount of desmosine conjugate can be an intermediate amount. In other words, too much capture conjugate or too little capture conjugate can shift the degree of competition in an undesirable way with respect to the sample desmosine. Thus, for a particular plate, the appropriate amount of capture desmosine can be selected to achieve a desmosine assay sensitivity and a corresponding higher slope of the standard curve within desired ranges. For the use of microtiter wells, extra unconjugated ovalbumin or other capture macromolecule can be combined with the capture conjugate to block any unfilled sites on the well so that no nonspecific binding can occur of anti-desmosine antibody bound to sample desmosine.

As noted above, the anti-desmosine antibodies can be generated in an appropriate animal. The detection antibody with the enzyme label should have a high titer and/or binding affinity for the anti-desmosine antibody. For anti-desmosine antibodies raised in rabbits, the detection antibody can comprise a goat anti-rabbit (antibody) antibody. Other detection antibodies are commercially available. A suitable enzyme is conjugated to the detection antibody. An appropriate enzyme can react with a substrate to produce a detectable product. Goat anti-rabbit IgG conjugates with horseradish peroxidase as well as other detection antibodies are available from Vector Laboratories and Cell Signaling Technologies (MA, USA).

Alternatively, the detection antibody can be biotinylated, and the signal generating enzyme, e.g., horse radish peroxidase, can be conjugated to avidin or strepavidin. Biotin is a natural cofactor with a strong binding affinity to proteins avidin and streptavidin. Streptavidin and avidin bind with high specificity to biotin, allowing for the signal generation upon addition of the colorimetric substrate, e.g., TMB. Other similar detection antibody formats can be used as desired.

Suitable peroxidase enzyme substrates are available commercially. For example, Vector Laboratories (CA, USA) sells a range of suitable substrates. For example, 2,2′-azino-bis(3-ethylbenzthiazoline-6-sulfonic acid) reacts to form a green product and 3,3′,5,5′-tetramethyl benzidine (TMB) reacts to form a blue reaction product. In particular, TMB has a high sensitivity, which can be desirable for the ELISA assay described herein. For TMB, sulfuric acid can be used as a stop solution. After application of the stop solution, the TMB product becomes yellow and can be measured at 450 nm. Alternatively, no stop solution can be used, and the blue color product can be measured at 650 nm.

To obtain standard curves for evaluating assay results, the assay is performed with standard desmosine samples over an appropriate range of concentrations. The assay measurements, such as light absorption at the appropriate wavelength, are plotted as a function of concentration to generate a standard curve. The standard curve then can be used to evaluate patient samples to estimate the quantity of desmosine in the samples. In general, a set of desmosine standard solutions are prepared with a range of dilutions to cover an appropriate range for evaluating patient samples under the assay protocol. The desmosine standard solutions can be prepared from a stock desmosine. Desmosine in a chloride salt form is available from Elastin Products, which can be used to create the standard solutions with desired dilutions. In general, the standard solutions can cover a range in desmosine concentrations to deliver at the assay volume, for example, from about 0.1 pmole to about 64 picomoles and in further embodiments from about 1 pmole to about 32 pmoles desmosine. In general, a set of desmosine standard solutions can include at least 4 solutions, in further embodiments at least about 5 solutions, in some embodiments from 6 to 50 solutions, and in other embodiments, 7 to 25 solutions with appropriate spacing of concentrations over the selected range. A person of ordinary skill in the art will recognize that additional ranges of desmosine standard concentrations and numbers of solutions within the explicit ranges above are contemplated and are within the present disclosure. In further embodiments, the desmosine used to make the standard solutions can be further affinity purified to reduce possible matrix background effects. Dilution for the standard samples can use buffered saline or the like.

A kit for performing the assay can comprise the core components, i.e., the anti-desmosine antibody and the desmosine-capture macromolecule conjugate. Optionally, a kit can comprise one or more additional components for the assay as summarized above. Reagents can be supplied in at the desired dilution or at a known concentration that can then be further diluted to a desired dilution. For performing the assay, additional reagents are used, such as wash solutions, and the like, which generally are sufficiently pure and have appropriate compositions so as to not interfere with the assay. Wash solutions and the like can comprise purified water, saline, buffer, combinations thereof or the like, and these solutions are generally commercially available. A kit can comprise wash solutions if desired. A kit can further comprise instructions for the proper performance of the assay. The assay can be designed for performance on an automated system, as described further below, and the kit components can be appropriately packed for such an application. The components of the kit may or may not be physically packaged together, although packaging certain components, such as the core components may be convenient.

The assay can be designed to operate with a selected volume, and the reagent amounts can be scaled accordingly. In reasonable formats, the assay can use modest amounts of urine for the assay, such as from about 5 microliter (4) urine to about 200 μL, and in further embodiments from about 10 μL urine to about 100 μL urine. The assay can be correspondingly designed to measure desmosine levels from about 0.1 picomoles (pmol) to about 1000 pmol, in further embodiments, from about 0.2 pmol to about 500 pmol and in additional embodiments from about 0.25 pmol to about 200 pmol. A person of ordinary skill in the art will recognize that additional ranges of urine volumes and desmosine levels within the explicit ranges above are contemplated and are within the present disclosure.

In additional or alternative embodiments, standard solutions can be distributed for an assay site to generate a standard curve using the conditions at the particular facility. A single standard solution can be provided for dilution into a set of individual standard solutions. Standard solutions may or may not be packaged together as portions of a kit.

### Performance of the Assay

An integral part of performing the assay involves obtaining the samples. Standard samples are described above, and measurements on patient samples for diagnosis are, of course, the ultimate objective of the assay design. In some embodiments, as noted above, the urine samples from a patient can be introduced into the assay without significant dilution and/or without hydrolysis processing. The ELISA assay can then be performed on a sample using the reagents outlined above. The specific assay steps are described in detail below. The basic format of the ELISA assay described herein involves a competition for an anti-desmosine antibody between a sample's solubilized desmosine and desmosine bound to a capture medium. Based on the improved assay format and assay reagents described herein, the ability to process the sample desmosine with reduced processing of the sample urine simplifies the assay and reduces the assay time and cost, while not sacrificing the diagnostic ability of the assay.

Urine samples obtained from a patient can be transferred to an appropriate laboratory for performance of the assay. For example, urine samples can be obtained as a small aliquot from a 24 hour urine collection. A significant advantage of the ELISA assay described herein is the ability to introduce the samples into the assay without significant additional processing. The samples can be filtered or centrifuged to remove any solid contaminants. To perform acid hydrolysis, urine samples can be subjected to acid hydrolysis under acidic conditions with heat. For example, the urine can be combined with a strong acid at a fairly high concentration and then heated for several hours to more than 24 hours. However, in some embodiments of the ELISA of particular interest, the urine samples herein can be processed without acid hydrolysis.

Furthermore, to obtain desirable assay results, the urine samples can be diluted to balance urine matrix effects with assay sensitivity. Dilution can be used to reduce background matrix effects; although as described herein an improved ELISA assay can be performed without significant interference from matrix effects without additional efforts to reduce these if the samples are diluted appropriately. If the urine samples are not diluted sufficiently, the assay fails to achieve desirable results. However, if the urine samples are diluted more, the assay sensitivity drops to undesirable levels. Using the improved antibodies and conjugates described herein, the proper balance can be achieved for successful urine assays. The dilution can be achieved through the specific addition of buffer or through the dilution of added reactants to the assay with roughly the same effect. In some embodiments, the total dilution of the urine can be to achieve a total assay volume of about 1.3 times the urine volume to about 4 times the urine volume, in further embodiments from about 1.4 times the urine volume to about 3.5 times the urine volume, and in other embodiments from about 1.5 times the urine volume to about 3 times the urine volume. A person of ordinary skill in the art will recognize that additional ranges of assay volumes within the explicit ranges above are contemplated and are within the present disclosure.

As noted below, sample measurements can be normalized for creatinine levels, which may account for natural dilution of the urine from the patient, depending for example, on how much water or other liquids were consumed over the collection period. Generally, for urine samples not exhibiting significant dilution, the creatinine concentration is not used to perform the dilution. In contrast, Cocci et al., cited above, describe a dilution of a urine sample so that the creatinine level is below 20 μg per well. The preparation of urine samples under the current ELISA assay without acid hydrolysis and/or dilution based on creatinine concentration provides a significant savings in time and sample handling, such that the ELISA assay is significantly more suitable for performance as a commercial assay at a reasonable cost.

The prepared urine samples are then incubated with the anti-desmosine antibody and the capture desmosine conjugate. In some embodiments based on microtiter wells, the urine samples are combined with the anti-desmosine antibody in the assay well where the antibody can further contact the bound capture desmosine in the well. In further embodiments, the anti-desmosine antibody can be incubated with sample desmosine and capture desmosine conjugate, which may be attached to a bead or the like, and after a sufficient incubation time, the capture desmosine conjugate is immobilized so that the capture desmosine conjugate with bound anti-desmosine antibody can be separated from the sample desmosine. Following the selected incubation time and immobilization of the capture desmosine conjugate, the well is rinsed. The total incubation time involving the anti-desmosine antibody and the urine sample prior to rinsing the well can be in some embodiments, no more than about 6 hours, in further embodiments no more than about 5 hours, in additional embodiments no more than about 4 hours, and in other embodiments from about 30 minutes to about 3 hours. A person of ordinary skill in the art will recognize that additional ranges of incubation times within the specific ranges above are contemplated and are within the present disclosure. Based on the improved ELISA assay described herein, reduced incubation times and good results with desirable low matrix effects have been obtained with a single incubation of the urine sample and anti-desmosine antibody within the assay well.

After completing the incubation time on the assay well, the wells are rinsed to maintain the captured anti-desmosine antibodies and remove the sample desmosine and corresponding bound anti-desmosine antibody. In general, the rinsing can be performed a plurality of times, such as two times, three times, four times, five times, six times or more than six times. The rinse volume can be selected based on the well size by a person of ordinary skill in the art.

The washed wells can then be contacted with the detection antibody. The detection antibody is directly or indirectly (such as through an intermediate antibody) labeled with an enzyme. The detection antibody is generally incubated with the well holding the captured anti-desmosine antibody for a selected detection incubation time. In some embodiments, the detection incubation time can be no more than about 5 hours, in some embodiments, no more than about 4 hours and in additional embodiments from about 30 minutes to about 3 hours. The amount of capture antibody can be selected based on the size of the well and coating of the well by a person of ordinary skill in the art. A person of ordinary skill in the art will recognize that additional ranges of incubation time within the specific ranges above are contemplated and are within the present disclosure. At the end of the detection incubation time, the wells are rinsed to remove any unbound detection antibody. In general, the wells can be rinsed a plurality of times to remove unbound detection antibody, such as two times, three times, four times, five times, six times or more than six times. The volume of rinse liquid can be selected based on the volume of the wells.

Once the wells are prepared with the enzyme label, a substrate can be added to the wells. The substrate is incubated with the wells for a specific period of time, the development incubation time. An enzyme can act as an amplifier to increase the detectable product since the enzyme is not consumed but can react with additional substrate over time. Since the product of the substrate reacting with the enzyme is quantified, the incubation time with the substrate is relatively important since additional substrate reacts with the enzyme with the passage of time. Therefore, the plates should be read at the selected development incubation time to measure the detectable reaction product. In embodiments of particular interest, the reaction product turns a specific color, so that a light absorption measurement at a selected wavelength can be used to measure the reaction product. In alternative embodiments, a stop solution can be optionally added to the well at the end of the development incubation time to end further significant reaction of the substrate with the enzyme. A stop solution can then provide additional time to read the reaction product in the wells, if desired. In some embodiments, the stop solution can react with the product to change the measurement, but the measurement of the wells can be adjusted accordingly. Measurements of the color of the wells or other corresponding optical measurements can be made to obtain a measurement for a particular urine sample or standard sample. The samples can be run in multiple copies to obtain a more robust measurement from the corresponding average. For example, a sample can be run in two repeats, three repeats, four repeats or more than four repeats, as desired. An average of the duplicate sample measurements can then be carried forward for evaluation, e.g., formation of a standard curve using standard sample measurements or evaluation of a desmosine concentration for a patient sample.

In general, the urine sample measurements can be normalized according to the creatinine levels of the urine. The creatinine can be used as an approximate concentration factor so that the desmosine measurements can be more accurately reflect the condition of the patient. In particular, a urine sample can be obtained from a patient at different inherent dilution levels depending on the amount of liquids consumed by the patient over the relevant time period, so correction with normalizing to the creatinine level can be used to obtain values that are more diagnostic. However, if a disease condition alters the creatinine levels in patients, the normalization has the risk of biasing the results, so it may or may not be used. For evaluation of aneurysm risk based on desmosine levels, normalization by creatinine levels was found to be useful, as described in the example below. Creatinine levels can be measured in the urine using appropriate techniques, such as commercially available creatinine assays. For creatinine measurements, see for example, Chasson et al., “Determination of creatinine by Means of Automated Chemical Analysis,” Am. J. Clin. Pathol. Vol. 35, pp. 83-88 (1961), incorporated herein by reference.

The optical measurements from the urine sample assay are converted to desmosine levels using a standard curve generated by performing an equivalent assay with desmosine standards. In particular, the measured reading from a sample is found on the standard curve, and the corresponding desmosine concentration is found on the standard curve. This desmosine level or the adjusted value based on the normalized value for the amount of creatinine can then be incorporated into a diagnosis process. For example, the desmosine value obtained from the standard curve can be normalized based on a milligram of creatinine. In other words, values carried forward in the analysis can be picomoles desmosine per milligram creatinine (desmosine (pmoles)/creatinine (mg)), although other scaling would yield equivalent results just adjusted by a constant value.

### Determination of Aneurysm Risk and Diagnostic Use Generally

The assay measurements described herein can be used as a diagnostic tool for aneurysms. The results presented in the examples demonstrate the ability of a urine assay to provide meaningful diagnostic differentiation between a control group of patients and patients with aortic aneurysms. The ability to have a diagnostic tool that is non-invasive, relatively inexpensive and routine with respect to sample collection can lead to earlier diagnosis with a corresponding significant potential for improvement in outcomes and a corresponding reduction in total medical costs and social costs. Elevated desmosine levels have also been correlated with chronic obstructive pulmonary disease and rarer connective tissue diseases, and the availability of a commercial desmosine assay may also benefit the diagnosis and tracking of these diseases.

The results below demonstrate the ability to reasonably diagnose of an elevated probability of an aneurysm based on measurements of desmosine levels in urine. In this section, reference to a desmosine level may or may not be scaled by the amount of creatinine. As noted above, desmosine levels can also reflect to some degree iso-desmosine, but since iso-desmosine is also an elastin breakdown product, cross reaction with iso-desmosine in the assay generally is not detrimental and could be beneficial since overall desmosine moiety levels would be higher. The disease and control groups may not have completely separate levels of desmosine, but with the effective assays the extent of false negative reading and false positive readings are reasonable based on the analyses described herein.

Based on the measurements of control groups, an expected range of readings for a healthy individual can be set. An elevated reading above the normal range can then be flagged as an unusual reading. Based on a reading of an elevated desmosine level, additional tests can be performed on the patient to further the diagnosis. The cutoff of the normal range can be selected to balance the number of false negative readings and false positive readings. For example, selection of a lower cutoff of the “normal” range results in a larger number of false positive readings and a smaller number of false negative readings, and selection of a larger cutoff value of the “normal” range will result in a larger number of false negative readings and a smaller number of false positive readings. For example, a cutoff value can be set such that at least 75% of adult AAA aneurysm patients have a desmosine/creatinine concentration ratio greater than at least 75% of a healthy adult control population as measured by the ELISA assay, or in other embodiments a concentration ratio of 90% of a healthy adult control population. In some embodiments, the normal range can be selected with the upper cutoff of the normal range from about 50 picomoles desmosine/milligram creatinine (pmoles/mg) to about 675 pmoles/mg creatinine. A person of ordinary skill in the art will recognize that additional ranges of cutoff values within the explicit range above are contemplated and are within the present disclosure and that absolute values may vary with specific assay methods.

To reduce the number of false negative and false positive readings, the desmosine measurements can be considered in the context of additional measurements. For example, the use of desmosine measurements in combination with other markers has been described in published U.S. patent application 2009/0186370 to Ogle et al., entitled “Diagnostic Biomarkers for Vascular Aneurysm,” incorporated herein by reference. In particular, suitable additional markers include, for example, a collagen degradation product or a matrix metalloproteinase, such as MMP-2, MMP-9 or a combination thereof. The use of a matrix of results can be evaluated to determine whether or not additional follow up for aneurysm risk is appropriate.

If assays determine a likelihood of an aneurysm, appropriate follow up for the patient can be put into place. In particular, additional testing can then be performed, such as appropriate imaging, e.g., a duplex ultrasound, CT scan, MRI or the like. Also, appropriate treatment can be pursued. A desire for early diagnosis is furthered by the promise of effective therapies to arrest or reverse an aneurysm. Such potentially effective early therapies are described, for example, in U.S. Pat. No. 7,713,543 to Vyavahare et al., entitled “Elastin Stabilization of Connective Tissue,” incorporated herein by reference.

An elevated level of desmosine can also suggest other disease conditions for the patient. In particular, elevated levels of desmosine have been found in chronic obstructive pulmonary disease. See Cocci cited above. Thus, the determination of an elevated desmosine levels can be indicative of aneurysm, COPD or potentially other connective tissue disorders. The ELISA assay can be used to evaluate the Desmosine level, and the determination of an elevated desmosine level can then be used to further investigate the particular disease condition. As noted above, other blood tests may be combinable with the desmosine evaluation to provide further diagnostic ability.

### EXAMPLES

Desmosine and isodesmosine were obtained according to the procedure described in Starcher et al., Prep. Biochem. Biotecimol. 5 (5&6), 445-460 (1975) entitled “A Large Scale Procedure for Purification of Desmosine and Isodesmosine,” incorporated herein by reference. Since the following procedures apply equally to measurement of both isomers, detection of desmosine and isodesmosine is simply referred to as desmosine unless specifically indicated otherwise. Microtiter plates used were Corning Costar 9018 from Sigma unless indicated otherwise, and 1-ethyl-3-(3-dimethylaminopropyl)-3-carbodiimide (EDC), sodium cyanoborohydride, and sodium meta periodate were obtained from Thermo Fisher Scientific Inc. Peroxidase labeled goat anti-rabbit IgG, tetramethylbenzidine (TMB), wash solution, serum albumin blocking solution, and coating buffer were obtained from Kirkegaard & Perry Laboratories, Inc. Ovalbumin, 0.5 M potassium phosphate buffer pH 7.5, and 2-[N-morpholino]ethane sulfonic acid (MES) were obtained from Sigma. Urine samples were obtained by Vatrix Medical, Inc. Antibodies to the desmosines for RIA were made as previously discussed by Starcher and Scott in Atm. Clin. Biochem. 1992: 29:72-78, incorporated herein by reference.

### Example 1

### Synthesis of Desmosine-Protein Conjugate

Desmosine was conjugated to ovalbumin using 1-ethyl-3-(3-dimethylaminopropyl)-3-carbodiimide (EDC) as the coupling agent in conjugation buffer (0.1M MES (2-[N-morpholino]ethane sulfonic acid), pH 4.9).

Ovalbumin (10 mg) was dissolved in 1 mL of conjugation buffer followed by the addition of desmosine (1.0 mg) to form a reaction mixture, into which 100 μL freshly made aqueous EDC solution (100 mg/mL) was added to initiate the coupling process. The reaction mixture was incubated for 2 hr at room temperature (RT) and then quenched by the addition of ethanolamine (1M, pH 9.8) to a final concentration of 50 mM ethanolamine. The excess ethanolamine and unbound desmosine was removed from the desmosine-ovalbumin (DOV) conjugate by dialyzing the quenched reaction mixture overnight against several changes of deionized water.

### Example 2

### Formation of High Affinity Capture Plate

To form a high affinity capture plate, the desmosine-ovalbumin (DOV) conjugate from Example 1 was used to coat the wells of a 96 well microtiter plate. Specifically, the DOV conjugate from example 1 was dissolved in a coating buffer (0.05 M carbonate buffer, pH 10) to give a coating solution with a concentration of 1 μg/mL. The coating solution (100 μL) was pipetted into each well of the microtiter plate and incubated at room temperature for 1 h followed by washing the wells (3 times) with wash solution 200-300 μL PBS containing 0.05% Tween 20, pH 7.8). The wells were then blocked by adding 1% bovine serum albumin (BSA, 200 μL) and incubated at room temperature for 1 hr. The wells of the microtiter plate were washed once more with the wash solution 200-300 μL) to form the high affinity capture plate.

### Example 3

### Preparation of Anti-Desmosine Antibody

Desmosine was linked to keyhole limpet hemocyanin (KLH) with glutaraldehyde yielding a KLH-desmosine conjugate that comprises about 8-12 moles of desmosine per mole of KLH. The KLH-desmosine conjugate (1 mg) was emulsified with 1 mL Fruends complete adjuvant and 1 mL saline and injected subcutaneously into rabbits. The rabbits was then boosted once a month with 100 mg of the KLH-desmosine conjugate and bled every 2 weeks after the initial injection. High titer serum usually started after 2-3 months and continued for up to a year.

Titer of the anti-desmosine antibody in the serum was measured by serially diluting the serum in PBS to form a set of diluted serum samples. The measurement of titer is described, for example, in the Immuno Assay Handbook, 3rd Edition, by David Geoffrey Wild, Elsevier Science, July 2005, incorporated herein by reference. Each of the diluted serum samples (100 μL) was then incubated in individual wells of the high affinity capture plate of example 2 for 1 hour followed by washing (3 times) each sample well with wash solution. Horseradish peroxidase (HRP) labeled goat anti-rabbit immunoglobulin G (IgG) diluted 1/5000 in 0.5 M potassium phosphate buffer containing 1% BSA, pH 7.8 (100 uL) was added to each sample well and incubated at room temperature for 1 hr. The plate was then washed (3 times) and tetramethylbenzidine (TMB, 100 μL) was added into each well, and the plate was incubated at room temperature for 10 min. The absorbance of the wells were measured on a titer plate reader (Molecular Devices Spectra Max Plus) at 650 nm and recorded for subsequent calculation. The equipment used for the ELISA analysis was a titer plate reader programmable to read a logit-log curve. The titer plate was first coated with the ovalbumin DES conjugate and antibody was serially diluted out to 1/5000 and reacted with the second antibody. The second reaction of the horseradish peroxidase was measured colormetrically.

High titer serum from individual rabbits was pooled separately to form individual lot of serum and freeze dried in 0.5 ml aliquots. Each lot of serum was analyzed, using the procedure disclosed above to determine binding characteristics in order to maintain uniform assays.

### Example 4

### General ELISA Format and Formation of Standard Curve

The general ELISA format is described in the context of the generation of a standard curve using purified desmosine standards.

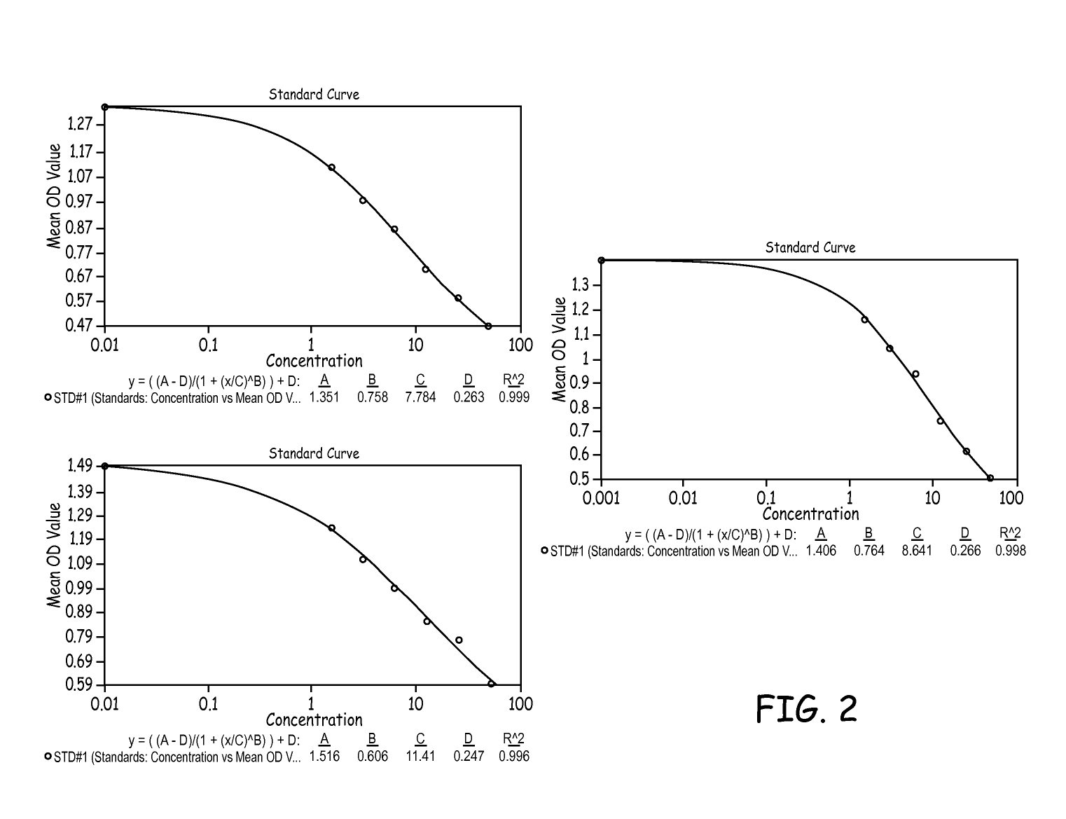

Desmosine (DES) standards were obtained from Elastin Products that have been calibrated on an amino acid analyzer. All desmosine stock solutions and standards were stored in siliconized glass containers. The desmosine stock solution was serially diluted in 0.5 M sodium phosphate buffer (pH 7.8) to give desmosine standard samples with concentration values ranging between 1 and 50 picomoles in 50 μL. The standard curve was prepared by incubating 50 μL of the anti-desmosine antibody (the primary antibody) from Example 3 at an initial concentration of around 70 mg/ml diluted 1/5000 in 0.5 M potassium phosphate buffer, pH 7.8 with each of the desmosine standard samples (50 μL) in individual wells of the DOV coated high affinity capture plate from example 2 for a final dilution of 1/10,000. After incubating for 1 hour, each of the samples wells were washed (3 times) with wash solution. HRP labeled goat anti-rabbit immunoglobulin G (IgG) diluted 1/5000 in 0.5 M potassium phosphate buffer containing 1% BSA, pH 7.8 (100 uL) was added to each sample well and incubated at room temperature for 1 hr. The plate was then washed (3 times) and tetramethylbenzidine (TMB, 100 μL) was added into each well, and the plate was incubated at room temperature for 10 min. The absorbance of the wells were measured on a titer plate reader (Molecular Devices Spectra Max Plus) at 650 nm and recorded. The equipment used for the ELISA analysis was a titer plate reader programmable to read a logit-log curve. The recorded absorbance data were correlated with the desmosine standard concentration to form a standard curve, which is a plot of the absorbance as a function of desmosine concentration. Three standard curves are plotted in FIG. 2 showing logit-log curves, and all were valid for measuring between 1-20 pmoles DES.

Standard checkerboard schemes adapted for the present assay were used to optimize the saturation curve for the plate coating by plotting desmosine-ovalbumin (DOV) concentrations vs. primary antigen concentration and to optimize the secondary antibody concentration by plotting primary antibody (goat anti-rabbit HRP) concentration against second antibody concentration keeping the optimal level of DOV constant. Conditions were adjusted to obtain a desired level of ELISA color development of 1.0-1.5 for the zero level of desmosine following 10 minutes incubation at room temperature. Adjustments were made to establish an ELISA standard curve that read accurately between 0.5 and 50 picomoles of desmosine.

### Example 5

### Enzyme-Linked Immunosorbent Assay (ELISA) for Urine Sample and Matrix Effect Testing

The performance of the ELISA assay for patient urine samples is described in the context of evaluating matrix effects in the urine samples.

Urine samples (50 μL) as obtained from a healthy patient or diluted with added desmosine were incubated with 50 μL of the anti-desmosine antibody (the primary antibody) from Example 3 with an initial concentration of about 1 mg/ml diluted 1/4000 in 0.5 M potassium phosphate buffer, pH 7.8 in individual well of the DOV coated high affinity capture plate from Example 2. After incubating for 1 hour, the sample well was washed (3 times) with wash solution. HRP labeled goat anti-rabbit immunoglobulin G (IgG) initially 1 mg/ml diluted 1/5000 in 0.5 M potassium phosphate buffer containing 1% BSA, pH 7.8 (100 uL) was added to the sample well and incubated at room temperature for 1 hr. The well was then washed (3 times), and tetramethylbenzidine (TMB, 100 μL) was added into the well, and the plate was incubated at room temperature for 10 min. The absorbance of the well was measured on a titer plate reader (Molecular Devices Spectra Max Plus) at 650 nm and recorded. The equipment used for the ELISA analysis was a titer plate reader programmable to read a logit-log curve. The recorded absorbance was compared with the standard curve from Example 4 to estimate the amount of desmosine in the urine sample.

This comparison testing was based on Clinical and Laboratory Standards Institute CLSI EP14-A2 Evaluation of Matrix Effects; Approved Guideline—Second Edition, incorporated herein by reference. Normal urine samples from 2 normal (or healthy) donors were collected and tested for desmosine moieties using the ELISA method above. The urine samples #1 and #2 were tested as is and then were then spiked with desmosine in triplicate. The amounts of added desmosine was at 2.5, 5, 7.5 10, 15 and 20 picomoles (i.e. amount of desmosine in 50 μL of urine). The results were compared with 5 control phosphate buffer samples containing from 2.5 to 20 picomoles of desmosine prepared as described above for the urine samples to determine if there are any urine matrix effects. The matrix effects data summary is listed in Table 1 below, which also lists coefficients of variation (CV) from the mean value.

The average different between the urine and phosphate buffer was 1.1 pmoles with a standard deviation of 3.3 pmoles, and there was a negative bias below 10 pmoles DES and a positive bias above 10 pmoles DES for urine versus phosphate buffer. Thus, this matrix effects test data shows a slight bias between urine and phosphate buffers, but the overall difference is small and acceptable.

TABLE 1

| | | Average | Stdev | | | |
|---|---|---|---|---|---|---|
| | | measured | measured | | | |
| | Measured | pmoles | pmoles | Measured | Theor. | Delta |
| sample | pmoles DES | DES | DES | % CV | DES | DES |
| urine #1 | 0.895/1.034/1.462 | 1.130 | 0.296 | 26.1% | NA | NA |
| urine #1 + 2.5 pmoles | 1.900/1.850/2.160 | 1.970 | 0.166 | 8.4% | 3.630 | 1.660 |
| urine #1 + 5 pmoles | 3.35/3.71/3.38 | 3.480 | 0.200 | 5.7% | 6.130 | 2.650 |
| urine #1 + 7.5 pmoles | 6.34/7.11/6.23 | 6.560 | 0.479 | 7.3% | 8.630 | 2.070 |
| urine #1 + 10 pmoles | 8.3/9.15/9.84 | 9.097 | 0.771 | 8.5% | 11.130 | −2.034 |
| urine #1 + 15 pmoles | 16.31/16.03/17.64 | 16.660 | 0.860 | 5.2% | 16.130 | 0.530 |
| urine #1 + 20 pmoles | 21.89/25.03/26.55 | 24.490 | 2.376 | 9.7% | 21.130 | 3.360 |
| urine #2 | 5.58/6.57/6.24 | 6.130 | 0.504 | 8.2% | NA | NA |
| urine #2 + 2.5 pmoles | 2.181/2.576/2.606 | 2.454 | 0.237 | 9.7% | 8.630 | −6.176 |
| urine #2 + 5 pmoles | 4.45/4.95/5.17 | 4.857 | 0.369 | 7.6% | 11.130 | −6.273 |
| urine #2 + 7.5 pmoles | 8.34/10.1/9.21 | 9.217 | 0.880 | 9.5% | 13.630 | −4.413 |
| urine #2 + 10 pmoles | 11.7/12.9/13.5 | 12.700 | 0.917 | 7.2% | 16.130 | −3.430 |
| urine #2 + 15 pmoles | 23.9/22.9/22.4 | 23.067 | 0.764 | 3.3% | 21.130 | 1.937 |
| urine #2 + 20 pmoles | 34.7/35.9/39.8 | 36.800 | 2.666 | 7.2% | 26.130 | 10.670 |
| Buffer + 2.5 pmoles | 2.27/2.1/1.97 | 2.113 | 0.150 | 7.1% | 2.5 | 0.387 |
| Buffer + 5 pmoles | 4.1/4.4/3.64 | 4.047 | 0.383 | 9.5% | 5 | 0.953 |
| Buffer + 7.5 pmoles | 7/5.7/6.5 | 6.400 | 0.656 | 10.2% | 7.5 | 1.100 |
| Buffer + 10 pmoles | 8.36/5.5/10.3 | 8.053 | 2.415 | 30.0% | 10 | 1.947 |
| Buffer + 15 pmoles | 15.3/14.6/13.5 | 14.467 | 0.907 | 6.3% | 15 | 0.533 |
| Buffer + 20 pmoles | 18.8/17.3/16.4 | 17.500 | 1.212 | 6.9% | 20 | 2.500 |

### Example 6

### Interference Testing with Added Isodesmosine

This comparison testing was based on CLSI EP7-A2 Interference Testing in Clinical Chemistry; Approved Guideline—Second Edition, incorporated herein by reference. This testing was conducted based on the ELISA assay described in Example 5 using low (around 2.5 pmoles DES) and high levels (around 20 pmoles DES) in urine and adding in the same levels of IDES in urine since there are equal amounts of the two moieties in elastin and in urine as has been reported in literature. The range of DES in the samples were based on what has previously been reported for DES in urine when measured using ELISA [Cocci, Matsumoto, Osakabe, cited above]. An interference testing data summary is listed in Table 2 below.

There is strong interference of IDES on this assay, so the ELISA assay is not specific for DES over IDES but measures both of them fairly accurately with both present in equal amounts. Since elastin breakdown should yield equal moles of DES and IDES, this assay can be used to measure both, but results will be higher than other assays that are specific for DES. Since the goal of this assay is to measure elevated DES levels, it is acceptable to use the assay to measure DES/IDES since they should be elevated at comparable levels at the same time. Thus, this ELISA assay can be used to measure desmosine moieties (i.e., DES and IDES) simultaneously in urine.

TABLE 2

| | | ave | | | | | |
|---|---|---|---|---|---|---|---|
| | | pmole | Std | % | Delta | % | ave % |
| Sample # | pmole DES | DES | Dev | CV | DES | bias | bias |
| DES 20 pmoles | 18.880/17.345/27.004 | 21.076 | 5.191 | 24.63 | 1.076 | | |
| DES 10 pmoles | 10.150/13.702/13.068 | 12.307 | 1.894 | 15.39 | 2.307 | | |
| IDES 20 pmoles | 5.853/7.761/9.228 | 7.614 | 1.692 | 22.23 | 7.614 | | |
| IDES 10 pmoles | 3.944/4.045/4.116 | 4.035 | 0.086 | 2.14 | 4.035 | | |
| DES/IDES | 44.948/45.114/46.366 | 45.476 | 0.775 | 1.70 | 25.476 | 227.38 | 201.92 |
| 20 pmoles | | | | | | | |
| DES/IDES | 21.671/20.975/27.666 | 23.437 | 3.679 | 15.70 | 13.437 | 134.37 | 119.38 |
| 10 pmoles | | | | | | | |
| DES/IDES | 11.837/11.633/15.384 | 12.951 | 2.109 | 16.29 | 7.951 | 159.03 | 177.26 |
| 5 pmoles | | | | | | | |
| DES/IDES | 7.441/7.168/9.281 | 7.963 | 1.149 | 14.43 | 5.463 | 218.53 | 202.46 |
| 2.5 pmoles | | | | | | | |

### Example 7

### Precision Testing

The precision testing was based on CLSI EP5-A2 Evaluation of Precision Performance of Quantitative Measurement Methods; Approved Guideline-Second Edition, incorporated herein by reference. Precision testing entailed preparation of 5 levels of desmosine in human urine. These samples were prepared as follows: Normal urine samples from 2 healthy donors were collected and tested for desmosine moieties using ELISA detailed in Example 5 above. The urine samples were tested in triplicate as is and with added desmosine. Samples with added desmosine had a selected amount of added desmosine ranging from 2.5 picomoles to up to 40 picomoles (where 40 picomoles is approximate amount of DES in 25 μL of urine) where other values of desmosine were made by diluting the 40 picomoles DES with various amounts of urine.

The Intra Assay Precision was tested first using the procedure below. The 5 levels of desmosine in human urine prepared above were tested, each one 10 times on one day and 1 lot of test kits using ELISA (i.e. 50 samples total). There were a couple of samples that were only tested 9 times due to availability of spaces in the 96 well plates but this was believed to be an adequate number of replicates to determine intra assay precision. In this intra assay precision or repeatability testing, the “closeness of agreement between independent test/measurement results obtained under stipulated conditions” was evaluated. To minimize variability, samples were usually run on the same day. In the referenced “CDER Reviewer Guidance Validation of Chromatographic Methods” document from the FDA on p. 13 the repeatability testing (i.e. intra assay precision) was performed on the same day. The intra assay precision testing data summary is listed in Table 3 below.

TABLE 3

| Sample # | pmole DES | ave pmole DES | Std Dev | % CV | Delta |
|---|---|---|---|---|---|
| urine #2 10 ul | 1.167/0.488/1.33/1.296/1.348/ | 1.1502 | 0.3281 | 28.53 | NA |
| | 0.973/1.728/1.12/1.168/0.884 | | | | |
| urine #2 10 ul + 5 pmol DES | 3.425/1.986/4.140/4.600/3.718/ | 3.6113 | 0.74618 | 20.66 | −2.539 |
| | 3.590/3.939/4.099/3.826/2.790 | | | | |
| urine #2 10 ul + 10 pmol DES | 9.312/6.835/11.945/11.275/10.972/ | 9.601 | 1.7407 | 18.13 | −1.549 |
| | 10.776/9.147/8.260/7.885 | | | | |
| urine #2 10 ul + 15 pmol DES | 15.768/12.625/18.004/21.686/16.995/ | 15.876 | 3.09798 | 19.51 | −0.274 |
| | 16.676/14.141/15.909/11.079 | | | | |
| urine #2 10 ul + 20 pmol DES | 22.267/16.718/20.21/24.228/20.025/ | 20.179 | 2.0964 | 10.39 | −0.972 |
| | 19.721/20.165/19.232/19.041 | | | | |
| | | | ave | 19.44 | |

The Inter Assay Precision test was also carried out with the 5 levels of desmosine in human urine prepared above. Each sample was tested 3 times on 10 different days and 1 lot of test kits using ELISA procedure outline in example 5 above (i.e.150 samples total). The inter assay precision data summary is listed in Table 4a-4j below. The average % CV of inter assay precision is 21.16%, which is less than the specified 25%.

TABLE 4a

| | | ave | | | Calc. | Theor. | Delta |
|---|---|---|---|---|---|---|---|
| | | pmole | Std | % | pmole | pmole | pmole |
| Sample # | pmoles DES | DES | Dev | CV | DES | DES | DES |
| urine #84 | 2.842/4.605/4.712 | 4.053 | 1.050 | 25.91 | | | |
| urine #84 10 ul + | 4.037/4.313/7.057 | 5.136 | 1.670 | 32.51 | 1.1 | 2.5 | −1.4 |
| 2.5 pmol DES | | | | | | | |
| urine #84 10 ul + | 8.015/6.071/9.252 | 7.779 | 1.604 | 20.61 | 3.7 | 5 | −1.3 |
| 5 pmol DES | | | | | | | |
| urine #84 10 ul + | 11.288/14.009/15.628 | 13.642 | 2.193 | 16.08 | 9.6 | 10 | −0.4 |
| 10 pmol DES | | | | | | | |
| urine #84 10 ul + | 26.743/36.114/40.195 | 34.351 | 6.897 | 20.08 | 30.3 | 20 | 10.3 |
| 20 pmol DES | | | | | | | |
| urine #84 10 ul + | 67.772/81.888/50.791 | 66.817 | 15.570 | 23.30 | 62.8 | 40 | 22.8 |
| 40 pmol DES | | | | | | | |
| | | | ave | 23.08 | | | |

TABLE 4b

| | | ave | | | Calc. | Theor. | Delta |
|---|---|---|---|---|---|---|---|
| | | pmole | Std | | pmole | pmole | pmole |
| Sample # | pmoles DES | DES | Dev | % CV | DES | DES | DES |
| urine #84 | 0.181/0.032/0.098 | 0.104 | 0.075 | 72.02 | | | |
| urine #84 10 ul + | 6.331/7.641/8.779 | 7.584 | 1.225 | 16.15 | 7.5 | 2.5 | 5.0 |
| 2.5 pmol DES | | | | | | | |
| urine #84 10 ul + | 9.372/9.687/9.943 | 9.667 | 0.286 | 2.96 | 9.6 | 5 | 4.6 |
| 5 pmol DES | | | | | | | |
| urine #84 10 ul + | 17.277/20.428/20.773 | 19.493 | 1.927 | 9.88 | 19.4 | 10 | 9.4 |
| 10 pmol DES | | | | | | | |
| urine #84 10 ul + | 33.433/30.624/30.444 | 31.500 | 1.676 | 5.32 | 31.4 | 20 | 11.4 |
| 20 pmol DES | | | | | | | |
| urine #84 10 ul + | 50.56/51.919/67.333 | 56.604 | 9.316 | 16.46 | 56.5 | 40 | 16.5 |
| 40 pmol DES | | | | | | | |
| | | | ave | 20.47 | | | |

TABLE 4c

| | | ave | | | Calc. | Theor. | Delta |
|---|---|---|---|---|---|---|---|
| | | pmoles | Std | % | pmole | pmole | pmole |
| Sample # | pmoles DES | DES | Dev | CV | DES | DES | DES |
| urine #84 | 1.391/1.606/1.48 | 1.492 | 0.108 | 7.24 | | | |
| urine #84 10 ul + | 5.486/5.89/7.063 | 6.146 | 0.819 | 13.33 | 4.7 | 2.5 | 2.2 |
| 2.5 pmol DES | | | | | | | |
| urine #84 10 ul + | 9.093/14.557/10.570 | 11.407 | 2.826 | 24.78 | 9.9 | 5 | 4.9 |
| 5 pmol DES | | | | | | | |
| urine #84 10 ul + | 20.495/25.850/18.331 | 21.559 | 3.871 | 17.95 | 20.1 | 10 | 10.1 |
| 10 pmol DES | | | | | | | |
| urine #84 10 ul + | 37.98/45.400/38.973 | 40.784 | 4.028 | 9.88 | 39.3 | 20 | 19.3 |
| 20 pmol DES | | | | | | | |
| urine #84 10 ul + | 70.199/61.868/77.828 | 69.965 | 7.983 | 11.41 | 68.5 | 40 | 28.5 |
| 40 pmol DES | | | | | | | |
| | | | ave | 14.10 | | | |

TABLE 4d

| | | ave | | | Calc. | Theor. | Delta |
|---|---|---|---|---|---|---|---|
| | | pmole | Std | % | pmole | pmole | pmole |
| Sample # | pmoles DES | DES | Dev | CV | DES | DES | DES |
| urine #84 | 5.109/5.808/1.795 | 4.237 | 2.144 | 50.59 | | | |
| urine #84 10 ul + | 6.842/5.404/5.404 | 5.883 | 0.830 | 14.11 | 1.6 | 2.5 | −0.9 |
| 2.5 pmol DES | | | | | | | |
| urine #84 10 ul + | 7.65/8.884/5.892 | 7.475 | 1.504 | 20.11 | 3.2 | 5 | −1.8 |
| 5 pmol DES | | | | | | | |
| urine #84 10 ul + | 16.587/13.677/12.529 | 14.264 | 2.092 | 14.66 | 10.0 | 10 | 0.0 |
| 10 pmol DES | | | | | | | |
| urine #84 10 ul + | 37.907/46.057/16.495 | 33.486 | 15.269 | 45.60 | 29.2 | 20 | 9.2 |
| 20 pmol DES | | | | | | | |
| urine #84 10 ul + | 78.92/67.335/28.318 | 58.191 | 26.511 | 45.56 | 54.0 | 40 | 14.0 |
| 40 pmol DES | | | | | | | |
| | | | ave | 28.53 | | | |

TABLE 4e

| | | ave | | | Calc. | Theor. | Delta |
|---|---|---|---|---|---|---|---|
| | | pmole | Std | % | pmole | pmole | pmole |
| Sample # | pmoles DES | DES | Dev | CV | DES | DES | DES |
| urine #84 | 1.517/1.504/1.478 | 1.500 | 0.020 | 1.32 | | | |
| urine #84 10 ul + | 4.373/4.983/4.937 | 4.764 | 0.340 | 7.13 | 3.3 | 2.5 | 0.8 |
| 2.5 pmol DES | | | | | | | |
| urine #84 10 ul + | 8.855/6.966/7.689 | 7.837 | 0.953 | 12.16 | 6.3 | 5 | 1.3 |
| 5 pmol DES | | | | | | | |
| urine #84 10 ul + | 13.785/13.533/10.98 | 12.766 | 1.552 | 12.16 | 11.3 | 10 | 1.3 |
| 10 pmol DES | | | | | | | |
| urine #84 10 ul + | 35.176/31.401/30.671 | 32.416 | 2.418 | 7.46 | 30.9 | 20 | 10.9 |
| 20 pmol DES | | | | | | | |
| urine #84 10 ul + | 66.165/57.664/59.612 | 61.147 | 4.454 | 7.28 | 59.6 | 40 | 19.6 |
| 40 pmol DES | | | | | | | |
| | | | ave | 7.92 | | | |

TABLE 4f

| | | ave | | | Calc. | Theor. | Delta |
|---|---|---|---|---|---|---|---|
| | | pmole | Std | % | pmole | pmole | pmole |
| Sample # | pmoles DES | DES | Dev | CV | DES | DES | DES |
| urine #84 | 1.906/2.735/3.424 | 2.688 | 0.760 | 28.27 | | | |
| urine #84 10 ul + | 6.378/9.093/9.303 | 8.258 | 1.632 | 19.76 | 5.6 | 2.5 | 3.1 |
| 2.5 pmol DES | | | | | | | |
| urine #84 10 ul + | 6.807/18.156/10.373 | 11.779 | 5.804 | 49.27 | 9.1 | 5 | 4.1 |
| 5 pmol DES | | | | | | | |
| urine #84 10 ul + | 13.897/19.819/21.008 | 18.241 | 3.809 | 20.88 | 15.6 | 10 | 5.6 |
| 10 pmol DES | | | | | | | |
| urine #84 10 ul + | 28.229/35.000/36.111 | 33.113 | 4.266 | 12.88 | 30.4 | 20 | 10.4 |
| 20 pmol DES | | | | | | | |
| urine #84 10 ul + | 31.699/54.507/54.659 | 46.955 | 13.212 | 28.14 | 44.3 | 40 | 4.3 |
| 40 pmol DES | | | | | | | |
| | | | ave | 26.53 | | | |

TABLE 4g

| | | ave | | | Calc. | Theor. | Delta |
|---|---|---|---|---|---|---|---|
| | | pmole | Std | % | pmole | pmole | pmole |
| Sample # | pmoles DES | DES | Dev | CV | DES | DES | DES |
| urine #84 | 2.758/1.657/4.919 | 3.111 | 1.659 | 53.34 | | | |
| urine #84 10 ul + | 9.961/7.768/8.812 | 8.847 | 1.097 | 12.40 | 5.7 | 2.5 | 3.2 |
| 2.5 pmol DES | | | | | | | |
| urine #84 10 ul + | 9.712/11.492/10.797 | 10.667 | 0.897 | 8.41 | 7.6 | 5 | 2.6 |
| 5 pmol DES | | | | | | | |
| urine #84 10 ul + | 20.26/20.594/23.079 | 21.311 | 1.540 | 7.23 | 18.2 | 10 | 8.2 |
| 10 pmol DES | | | | | | | |
| urine #84 10 ul + | 34.695/36.253/34.627 | 35.192 | 0.920 | 2.61 | 32.1 | 20 | 12.1 |
| 20 pmol DES | | | | | | | |
| urine #84 10 ul + | 46.204/51.815/52.905 | 50.308 | 3.596 | 7.15 | 47.2 | 40 | 7.2 |
| 40 pmol DES | | | | | | | |
| | | | ave | 15.19 | | | |

TABLE 4h

| | | ave | | | Calc. | Theor. | Delta |
|---|---|---|---|---|---|---|---|
| | | pmole | Std | | pmole | pmole | pmole |
| Sample # | pmoles DES | DES | Dev | % CV | DES | DES | DES |
| urine #84 | 1.724/2.766/1.827 | 2.106 | 0.574 | 27.27 | | | |
| urine #84 10 ul + 2.5 pmol DES | 6.417/7.671/7.651 | 7.246 | 0.718 | 9.91 | 5.1 | 2.5 | 2.6 |
| urine #84 10 ul + 5 pmol DES | 11.658/10.521/17.566 | 13.248 | 3.782 | 28.55 | 11.1 | 5 | 6.1 |
| urine #84 10 ul + 10 pmol DES | 15.885/16.063/22.698 | 18.215 | 3.883 | 21.32 | 16.1 | 10 | 6.1 |
| urine #84 10 ul + 20 pmol DES | 30.835/37.327/40.187 | 36.116 | 4.792 | 13.27 | 34.0 | 20 | 14.0 |
| urine #84 10 ul + 40 pmol DES | 52.19/57.220/60.4 | 56.603 | 4.140 | 7.31 | 54.5 | 40 | 14.5 |
| | | | ave | 17.94 | | | |

TABLE 4i

| | | ave | | | Calc. | Theor. | Delta |
|---|---|---|---|---|---|---|---|
| | | pmole | Std | % | pmole | pmole | pmole |
| Sample # | pmoles DES | DES | Dev | CV | DES | DES | DES |
| urine #84 | 1.505/1.455/2.358 | 1.773 | 0.508 | 28.63 | | | |
| urine #84 10 ul + | 7.887/6.752/11.924 | 8.854 | 2.718 | 30.70 | 7.1 | 2.5 | 4.6 |
| 2.5 pmol DES | | | | | | | |
| urine #84 10 ul + | 21.253/8.471/19.981 | 16.568 | 7.041 | 42.50 | 14.8 | 5 | 9.8 |
| 5 pmol DES | | | | | | | |
| urine #84 10 ul + | 20.172/21.109/27.179 | 22.820 | 3.804 | 16.67 | 21.0 | 10 | 11.0 |
| 10 pmol DES | | | | | | | |
| urine #84 10 ul + | 27.052/31.993/38.087 | 32.377 | 5.528 | 17.07 | 30.6 | 20 | 10.6 |
| 20 pmol DES | | | | | | | |
| urine #84 10 ul + | 48.482/50.484/56.739 | 51.902 | 4.307 | 8.30 | 50.1 | 40 | 10.1 |
| 40 pmol DES | | | | | | | |
| | | | ave | 23.98 | | | |

TABLE 4j

| | | ave | | | Calc. | Theor. | Delta |
|---|---|---|---|---|---|---|---|
| | | pmole | Std | | pmole | pmole | pmole |
| Sample # | pmole DES | DES | Dev | % CV | DES | DES | DES |
| urine #84 | 0.821/2.895/0.113 | 1.276 | 1.446 | 113.28 | | | |
| urine #84 10 ul + | 3.528/4.657/5.269 | 4.485 | 0.883 | 19.69 | 3.2 | 2.5 | 0.7 |
| 2.5 pmol DES | | | | | | | |
| urine #84 10 ul + | 9.665/11.269/16.591 | 12.508 | 3.626 | 28.98 | 11.2 | 5 | 6.2 |
| 5 pmol DES | | | | | | | |
| urine #84 10 ul + | 16.583/15.798/18.120 | 16.834 | 1.181 | 7.02 | 15.6 | 10 | 5.6 |
| 10 pmol DES | | | | | | | |
| urine #84 10 ul + | 23.087/33.131/32.896 | 29.705 | 5.732 | 19.30 | 28.4 | 20 | 8.4 |
| 20 pmol DES | | | | | | | |
| urine #84 10 ul + | 35.767/41.316/48.107 | 41.730 | 6.180 | 14.81 | 40.5 | 40 | 0.5 |
| 40 pmol DES | | | | | | | |
| | | | ave | 33.85 | | | |

The Inter Lot Precision test was carried with the 5 levels of desmosine in human urine prepared above. Each sample was tested 3 times using 3 different operators on 3 different days and 3 lots of test kits using ELISA procedure outlined in the example 5 above (i.e. 45 samples total). The FDA 64 Guidance for Industry Validation of Analytical Procedures: Methodology July 1999 recommends that a minimum of 9 determinations covering the specified range for the procedure (e.g., 3 concentrations/3 replicates each) for precision on p. 9. The inter lot precision data summary is listed in Table 4c, 4 g, and 4e above, with average % CV of 14.10%, 15.19%, and 7.92% respectively, the overall average % CV is also less than the specified 25%.

### Example 8

### Linearity/Curve Fitting/Range Testing

This linearity/curve fitting testing was based on CLSI EP7-A2 Evaluation of the Linearity of Quantitative Measurement Procedures: A Statistical Approach; Approved Guideline—Second Edition, incorporated herein by reference. Five levels were deemed adequate to generate an acceptable curve fit as long as they spanned the range of interest and had a correlation coefficient (r) of at least 0.95. The FDA 64 Guidance for Industry Validation of Analytical Procedures: Methodology July 1999, incorporated herein by reference, recommended that a minimum of 5 concentrations be tested for linearity and that immunoassays typically have non-linear responses on p 7. Also that in this case, the analytical response should be described by an appropriate function of the concentration (amount) of an analyte in a sample. This data was compiled from the test samples used for the precision testing. It is not expected that either of these immunoassays would have a linear fit as they are inherently non-linear. Curve fitting methods were applied as needed to obtain the best non-linear fit. A logit-log curve was found to give the best fit for the data and is the curve fitting equation that is typically used for ELISA. See FIG. 2 for some representative logit-log calibration curves. Correlations were always greater than 0.98.

### Example 9

### Recovery

These samples were prepared starting with a baseline urine sample and spiking it with a high level of desmosine and then performing a series of serial dilutions using the zero calibration. This recovery testing was done to verify that the DES added to the urine could be accurately measured to verify that nothing in the assay was interfering with its ability to measure DES in urine. This recovery testing was performed only once using ELISA procedure outlined in the example 5 above rather than 3 times as specified in the protocol since this was deemed adequate to assess desmosine recovery. This testing was done on 5 samples in duplicate. The proposed recovery test methods were consistent with those recommended in the CDER Reviewer Guidance Validation of Chromatographic Methods document from the FDA on p. 8 for recovery studies and CLSI EP7-A2 Interference Testing in Clinical Chemistry: Approved Guideline—Second Edition. The recovery data summary is listed in Table 5 below.

TABLE 5

| | | | Ave | StDev | Ave | |
|---|---|---|---|---|---|---|
| Added | Measured | Measured | Meas. | DES | DES | % |
| DES | DES | DES | DES | Meas. | Calc. | Recovery |
| 0 | 2.38 | 2.66 | 2.52 | 0.20 | 0 | |
| 3.4 | 5.82 | 6.37 | 6.10 | 0.39 | 3.58 | 105 |
| 6.9 | 9.12 | 9.4 | 9.26 | 0.20 | 6.74 | 98 |
| 13.7 | 14.68 | 11.96 | 13.32 | 1.92 | 10.80 | 79 |
| 27.5 | 32.4 | 35 | 33.70 | 1.84 | 31.18 | 113 |
| 45 | 50 | 52.92 | 51.46 | 2.06 | 48.94 | 109 |
| | | | | | ave | 100.8 |

### Example 10

### Radioimmunoassay (RIA)

This example evaluates the statistical difference between urine samples obtained from a control group and an AAA group based on radioimmunoassay (RIA) measurements of desmosine in the urine sample. This example demonstrates with a significant patient group and reasonable control group that patients with AAA have a statistically higher desmosine level relative to a control group and that spread of distributions of the desmosine levels of the two groups provide for value for the measurement of desmosine levels as a diagnostic tool for finding patients with AAA. Comparisons between RIA desmosine measurements and ELISA desmosine measurements indicate that ELISA desmosine measurements can similarly serve as a diagnostic tool.

In general, RIA assays are based on a competition for antibody binding between the antigen, which is desmosine in the present case, and a radioactive labeled antigen, which is a [¹²⁵I] labeled desmosine in the present case. To perform the competition, a fixed antibody is mixed with a known quantity of the radiolabeled antigen and with an appropriate amount of the sample containing the sample antigen to be measured. Antigen that is not bound to the antibody is removed, and the amount of bound radioactivity is measured. Standard antigen samples with known concentrations are run in the RIA assay to establish a standard curve. A test sample from a patient containing an unknown quantity of the antigen can be evaluated then using the RIA assay and the established standard curve. RIA assays can be designed to be very sensitive. An exemplary RIA assay for desmosine is disclosed in Starcher et al, Connective Tissue Research 1980, Vol. 7, pp. 263-267 entitled “Radioimmunoassay for Desmosine”, incorporated herein by reference.

### Synthesis of [¹²⁵I] Labeled Desmosine

One milligram of desmosine (1.9 μM) was mixed at room temperature with 0.26 mg (1 μM) of N-succinimidyl-3-(4-hydroxyphenyl) propionate (Bolton-Hunter reagent or BH) in 0.1 mL of 50 mM borate buffer, pH 10 to form a BH-Des complex. The reaction was stopped after one hour by the addition of 2 μL of 6N HCl. The reaction mixture is then applied to a 1 mL syringe containing 0.3 mL of Dowex 50-8X, 200-400 mesh, and unreacted reagent eluted with 1 mL of distilled water. The BH-Des complex was eluted with 0.4 mL of 4N NH₄OH. After evaporation the complex was redissolved in 100 μL of 250 mM sodium phosphate buffer, pH 7.4 to form a BH-Des complex solution.

Ten microliters of the BH-Des complex solution were then mixed with 25 μL enzymobeads (BioRad) that comprises insoluble oxidant, 75 μL 1% β-D-glucose in 250 mM sodium phosphate buffer, pH 7.4, and 200 μCi of ¹²⁵I to radiolabel BH-Des complex based on the oxidative reaction discovered by Greenwood et al in Biochem. J. (1963) 89, 114 entitled “The Preparation of ¹³¹I-Labelled Human Growth Hormone of High Specific Radioactivity”, incorporated herein by reference. After 30 minutes at room temperature, the radio labeled BH-Des was separated from side products by chromatograph on a 1×28 cm column of Biogel P2, 200-400 mesh with 1M NaCl and 200 mM sodium phosphate, pH 6.8. The [¹²⁵I]-BH-Des was stored frozen at −70° C. in aliquots and diluted in assay buffer immediately before use.

### Desmosine Radioimmunoassay

Desmosine antibodies produced according to the procedure outline in Example 3 was affinity puried and attached to amine terminated magnetic particles according to the manufacturer's instructions (PerSeptive Diagnostics, Cambridge, Mass.) to form Magnetic antibody. The [¹²⁵I]-BH-Des synthesized above was diluted in 0.5 M potassium phosphate buffer pH 7.8 containing 1.25% powdered DMEM—Dulbecco's Modified Eagle Medium (DMEM, Sigma Chemical Co.) to prevent non specific adsorption and to form a solution that has 500 CPM/μL radioactivity. Desmosine containing sample (1-50 μL) was incubated in 200 μL of [¹²⁵I]-BH-Des (100,000 CPM) and 50 μL of the magnetic antibody in a tube overnight. The magnetic antibody used was sufficient to bind 30% of the total radioactivity counts. After the incubation, the tube containing the assay mixture is placed in a magnetic separating rack for 1 min and then inverted to remove the supernatant. The magnetic antibody captured desmosine or [¹²⁵I]-BH-Des was retained in the tube while the unbound desmosine in the sample as well as unbound [¹²⁵I]-BH-Des were removed. The magnetic particles were then washed 3 additional times with 0.02 M Bis TrisPropan buffer containing 0.02% Tween 20, allowing 1 min each time foe the particles to stick to the magnet before inverting the rack. The particles remained in the tube were then counted for radioactivity. Desmosine standards with known concentrations were used to form standard curves. Urine samples containing desmosine were then assayed and the concentration of desmosine contained in the urine samples were estimated based on the standard curve.

RIA Assays Results from Control and AAA Groups

A total of 40 RIA values for the AAA group and 39 for the Control group were obtained using the method disclosed above. These RIA values are assumed to be independent measurements for the purposes of the following analyses. The following tables 6-8 give the basic summary statistics for the RIA assay value by group along with a t-test to test for a difference in assay result between groups. As shown in Table 6, there is very little overlap between the groups with a minimum value of 60.7 for the AAA group and a maximum of 62.82 for the Control. The variances are statistically different between groups so the Satterthwaite test is used which gives an estimated difference in means of 86.2 with 95% confidence limits of (60.6, 111.8) indicating the assay results are significantly different between groups (p<0.001).

TABLE 6

| Group | N | Mean | Std Dev | Std Err | Minimum | Maximum |
|---|---|---|---|---|---|---|
| AAA | 40 | 118.1 | 78.0600 | 12.3424 | 60.7100 | 431.5 |
| Control | 39 | 31.9115 | 19.2554 | 3.0833 | 0 | 62.8200 |
| Diff (1-2) | | 86.2082 | 57.1772 | 12.8669 | | |

TABLE 7

| Group | Method | Mean | 95% CL Mean | | Std Dev | 95% CL Std Dev | |
|---|---|---|---|---|---|---|---|
| AAA | | 118.1 | 93.1549 | 143.1 | 78.0600 | 63.9437 | 100.2 |
| Control | | 31.9115 | 25.6696 | 38.1534 | 19.2554 | 15.7364 | 24.8160 |
| Diff (1-2) | Satterthwaite | 86.2082 | 60.5668 | 111.8 | | | |

TABLE 8

| Method | Variances | DF | t Value | Pr > \|t\| |
|---|---|---|---|---|
| Satterthwaite | Unequal | 43.845 | 6.78 | <.0001 |

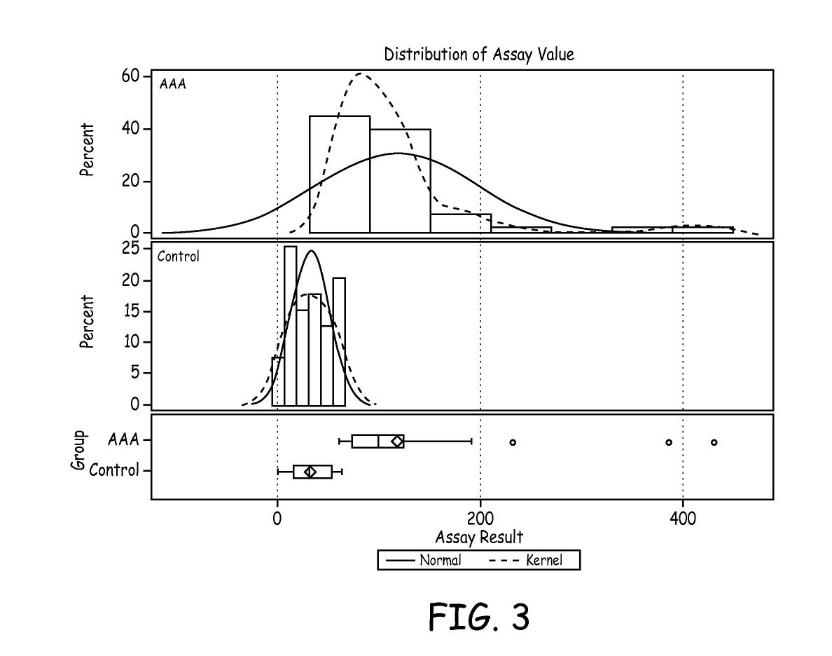

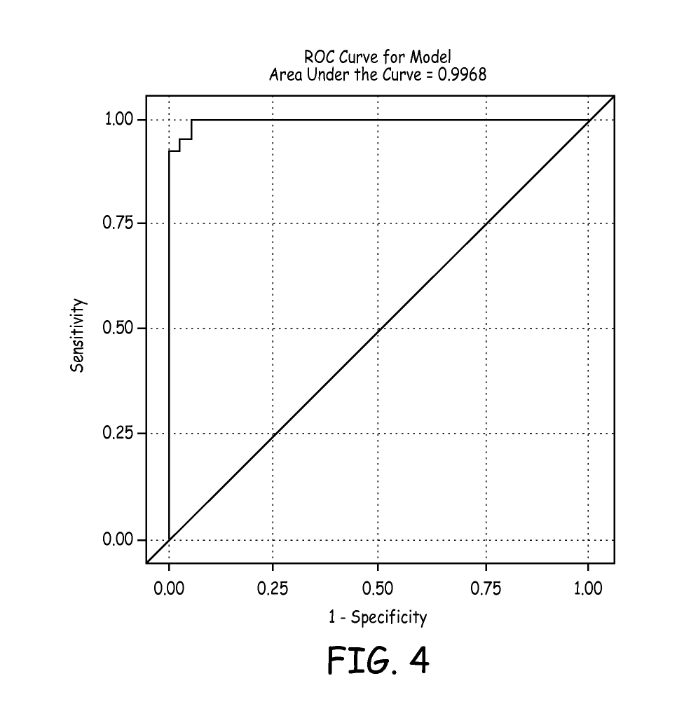

The distribution of RIA assay results by group is presented in the FIG. 3. It shows some skewness in the AAA group. Because of the differences in variance between groups and the skewness of the results in the AAA Group, a non-parametric Wilcoxon sign rank test was also conducted. This also demonstrates a significant difference between groups (p<0.0001). The 25%, 50% and 75% quartiles are given in Table 9 below. The area under the Receiver Operating Characteristic (ROC) curve from a logistic regression model of the assay value against AAA/Control is 0.997. The ROC curve is shown in FIG. 4.

TABLE 9

| | | Lower Quartile | Median Quartile | Upper Quartile |
|---|---|---|---|---|
| Group | N | 25% | 50% | 75% |
| AAA | 40 | 73.41 | 99.04 | 123.77 |
| Control | 39 | 15.30 | 30.87 | 52.73 |

### Example 11

### Comparison Testing

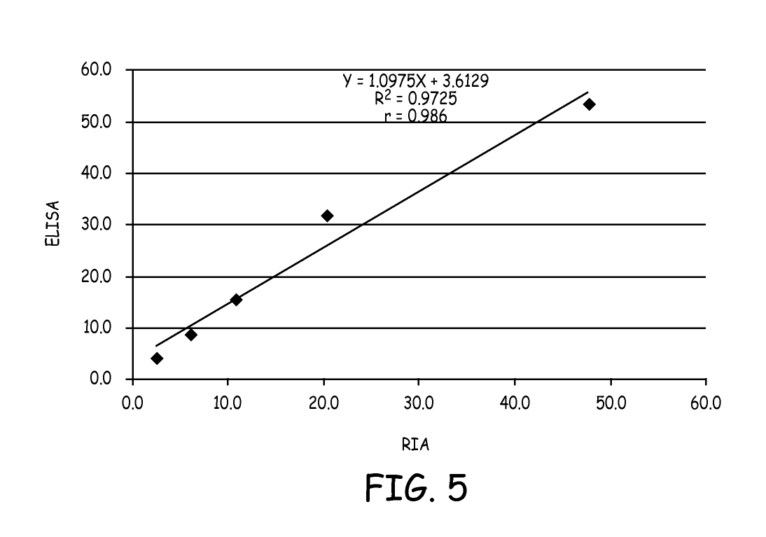

This comparison testing was based on CLSI EP9-A2 Method Comparison and Bias Estimation Using Patient Samples; Approved Guideline—Second Edition, incorporated herein by reference. The samples for comparison testing were the same samples that were used for the intra lot precision data for ELISA in Example 7, but involved testing the same 45 samples by RIA. The CLSI EP9-A2 protocol recommended that there were at least 40 samples be analyzed. The testing was conducted in a comparative fashion between an enzyme-linked immunoassay (ELISA) and a radioimmunoassay (RIA) to determine desmosine moiety levels in human urine. The comparison testing summary is listed in Table 10a-b below. Table 10a shows the RIA data, and Table 10b shows the difference between the RIA data in Table 10a and the average inter assay precision data from the ELISA assays. The RIA versus ELISA comparison testing was plotted and shown in FIG. 5. The isodesmosine interference to RIA assay was also performed and the results are listed in Table 10c below.

TABLE 10a

| | | ave | | | Calc. | Theor. | Delta |
|---|---|---|---|---|---|---|---|
| | | pmole | Std | | pmole | pmole | pmole |
| Comments | pmoles DES | DES | Dev | % CV | DES | DES | DES |
| urine #84 | 0.1/0.3/0.0/0.7 | 0.3 | 0.3 | 112.6 | | | |
| urine #84 10 ul + | 2.1/3.2/2.6/3.8 | 2.9 | 0.7 | 25.2 | 2.7 | 2.5 | 0.2 |
| 2.5 pmol DES | | | | | | | |
| urine #84 10 ul + | 5.5/8.9/6.0/5.0 | 6.4 | 1.7 | 27.5 | 6.1 | 5 | 1.1 |
| 5 pmol DES | | | | | | | |
| urine #84 10 ul + | 10.4/10.4/12.2/12.0 | 11.3 | 1.0 | 8.8 | 11.0 | 10 | 1.0 |
| 10 pmol DES | | | | | | | |
| urine #84 10 ul + | 23.6/22.7/21.5/14.5 | 20.6 | 4.1 | 20.1 | 20.3 | 20 | 0.3 |
| 20 pmol DES | | | | | | | |
| urine #84 10 ul + | 44.4/52.1/43.6/51.7 | 48.0 | 4.6 | 9.5 | 47.7 | 40 | 7.7 |
| 40 pmol DES | | | | | | | |

TABLE 10b

| | | ELISA all inter assay | RIA/ELISA | ELISA all inter assay | ELISA to RIA |
|---|---|---|---|---|---|
| Comments | Calc. pmole DES | Calc. pmole DES | Calc. pmole DES | Delta pmole DES | Delta pmole DES |
| urine #84 10 ul + 2.5 pmol DES | 2.7 | 4.5 | 59.1% | 2.0 | 1.8 |
| urine #84 10 ul + 5 pmol DES | 6.1 | 8.7 | 70.2% | 3.7 | 2.6 |
| urine #84 10 ul + 10 pmol DES | 11.0 | 15.7 | 70.0% | 5.7 | 4.7 |
| urine #84 10 ul + 20 pmol DES | 20.3 | 31.7 | 64.1% | 11.7 | 11.4 |
| urine #84 10 ul + 40 pmol DES | 47.7 | 53.8 | 88.6% | 13.8 | 6.1 |
| | | average | 70.4% | average | 5.3 |

TABLE 10c

| | | ave pmole | | | Delta | |
|---|---|---|---|---|---|---|
| Comments | pmole DES | DES | Std Dev | % CV | DES | % bias |
| IDES 2.5 pmoles | 0.1/0.5 | 0.3 | 0.3 | 0.9 | 0.0 | |
| IDES 5 pmoles | 0.9/0.0 | 0.5 | 0.3 | 1.4 | 0.2 | |
| IDES 10 pmoles | 0.1/0.8 | 0.5 | 0.5 | 1.1 | 0.2 | |
| IDES 20 pmoles | 1.1/3.4 | 2.3 | 1.6 | 0.7 | 2.0 | |
| IDES 40 pmoles | 2.6/2.8 | 2.7 | 0.1 | 0.1 | 2.4 | |
| IDES/DES 2.5 pmoles | 2.5/4.7 | 3.6 | 1.6 | 0.4 | 0.8 | 33 |
| IDES/DES 5 pmoles | 5.2/4.3 | 4.8 | 0.6 | 0.1 | −0.5 | −11 |
| IDES/DES 10 pmoles | 12.9/12.1 | 12.5 | 0.6 | 0.0 | 2.6 | 26 |
| IDES/DES 20 pmoles | 25.3/28.3 | 26.8 | 2.1 | 0.1 | 6.5 | 33 |
| IDES/DES 40 pmoles | 58.9/46.6 | 52.8 | 8.7 | 0.2 | 12.5 | 31 |
| | | | | | ave | 22.5% |

### Example 12

### Reference Interval Testing

This comparison testing was based on CLSI C28-A3 Guideline Defining, Establishing, and Verifying Reference Intervals in the Clinical Laboratory; Approved Guideline-Third Edition, incorporated herein by reference. A minimum of 125 urine samples from reference control individuals were tested using the ELISA procedure outline in example 5 above. This sample size was based on the minimum of 120 samples that was recommended in CLSI C-28-A3 protocol for reference interval testing. The reference intervals can be used to define levels to distinguish a potential disease state by variation from the reference values. There were 3 outliers found in the 125 reference urine samples analyzed as defined as greater than the average (291 pmoles DES/mg C) plus 3× standard deviation (124 pmoles DES/mgC) or >664 pmoles DES/mg C. These outliers were sample 25 with 694 pmoles DES/mg C, sample 92 with 1045 pmoles\DES/mgC and sample 113 with 793 pmoles DES/mg C. The reference interval data summary is listed in Table 11 below.

TABLE 11

| | | | | mg | pm | pm | |
|---|---|---|---|---|---|---|---|
| Sample # | Initial | Pertinent history | Age | C/ml | DES/ml | DES/mgC | M/F |
| 1 | MEI | | 30 | 0.43 | 145 | 337 | F |
| 2 | JI | heart disease & AAA family history | 29 | 0.76 | 154 | 203 | M |
| 3 | CAP | heart problems | 43 | 1.49 | 362 | 243 | M |
| 4 | JS | smokes | 55 | 1.08 | 280 | 259 | M |
| 5 | MM | | 52 | 0.83 | 197 | 237 | M |
| 6 | TL | | 49 | 0.65 | 350 | 540 | M |
| 7 | LML | father-stent | 43 | 1.26 | 143 | 113 | F |
| 8 | RD | | 30 | 1 | 152 | 152 | M |
| 9 | EA | | 42 | 0.86 | 188 | 219 | M |
| 10 | LA | | 41 | 0.8 | 200 | 251 | F |
| 11 | RD | | 49 | 1.62 | 359 | 222 | F |
| 12 | AD | | 18 | 1.59 | 671 | 422 | M |
| 13 | AD | | 23 | 1.82 | 538 | 296 | M |
| 14 | BS | | 46 | 1.51 | 429 | 284 | M |
| 15 | JC | | 45 | 0.76 | 249 | 327 | F |
| 16 | JC | | 46 | 1.23 | 325 | 263 | M |
| 17 | BCK | | 19 | 1.77 | 441 | 249 | M |
| 18 | LJS | | 19 | 1.44 | 531 | 369 | F |
| 19 | SAS | | 19 | 1.52 | 491 | 323 | F |
| 20 | TMW | | 32 | 2.22 | 766 | 345 | F |
| 21 | SMS | | 50 | 0.57 | 158 | 278 | F |
| 22 | DFS | | 53 | 1.78 | 985 | 553 | M |
| 23 | EB | | 47 | 1.08 | 280 | 258 | F |
| 24 | RMB | prostate cancer | 46 | 1.78 | 754 | 424 | M |
| 25 | PB | breast cancer | 47 | 1.88 | 1307 | 694 | F |
| 26 | FMD | | 68 | 2.23 | 391 | 175 | F |
| 27 | DA | | 46 | 1.09 | 124 | 114 | F |
| 28 | KE | | 53 | 0.62 | 110 | 177 | F |
| 29 | PAO | diabetes | 46 | 0.77 | 206 | 267 | F |
| 30 | JMS | | 27 | 1.25 | 280 | 224 | F |
| 31 | JN | | 48 | 1.45 | 347 | 239 | M |
| 32 | DZ | | 54 | 1.17 | 497 | 425 | F |
| 33 | VMA | | 41 | 1.76 | 388 | 220 | F |
| 34 | VB | | 55 | 1.54 | 337 | 219 | M |
| 35 | RB | | 28 | 0.98 | 266 | 271 | M |
| 36 | MR | diabetes in family | 26 | 1.15 | 277 | 241 | M |
| 37 | ST | | 45 | 1.05 | 186 | 177 | M |
| 38 | RT | hypertension meds | 44 | 0.24 | 46 | 191 | M |
| 39 | SZ | | 40 | 1.96 | 284 | 145 | M |
| 40 | BS | | 71 | 1.82 | 470 | 258 | M |
| 41 | LVMR | | 53 | 1.82 | 335 | 184 | M |
| 42 | MBS | SSRI meds | 43 | 2.13 | 757 | 355 | M |
| 43 | SK | | 33 | 1.16 | 440 | 379 | M |
| 44 | SA | | 26 | 1.21 | 214 | 177 | M |
| 45 | KRG | | 31 | 0.86 | 224 | 261 | M |
| 46 | MZ | smokes, family history of heart | 57 | 1.28 | 430 | 336 | F |
| | | disease | | | | | |
| 47 | RM | | 37 | 1.78 | 685 | 385 | M |
| 48 | LM | | 38 | 2.61 | 1070 | 410 | F |
| 49 | JB | | 28 | 0.93 | 518 | 557 | F |
| 50 | DB | | 30 | 1.78 | 593 | 333 | M |
| 51 | MS | family history of autoimmune | 36 | 0.87 | 457 | 525 | F |
| | | disorders | | | | | |
| 52 | JRG | | 26 | 2.96 | 1042 | 352 | M |
| 53 | MAS | | 37 | 1.87 | 522 | 279 | M |
| 54 | PN | | 47 | 2.35 | 454 | 193 | M |
| 55 | SM | | 38 | 2.36 | 153 | 65 | M |
| 56 | KIW | | 29 | 1.15 | 446 | 388 | F |
| 57 | MO | | 37 | 1.61 | 638 | 396 | M |
| 58 | KAB | | 26 | 1.54 | 557 | 362 | F |
| 59 | MDB | | 24 | 1.86 | 671 | 361 | M |
| 60 | WIT | family history of cancer | 47 | 1.14 | 176 | 154 | M |
| 61 | MMO | family history of aortic | 25 | 1.1 | 472 | 429 | M |
| | | aneurysms | | | | | |
| 62 | ZZ | | 30 | 1.91 | 275 | 144 | M |
| 63 | SP | | 33 | 1.28 | 125 | 98 | M |
| 64 | YB | | 37 | 0.53 | 63 | 119 | M |
| 65 | RLD | | 47 | 0.39 | 85 | 219 | F |
| 66 | DD | | 49 | 1.66 | 319 | 192 | M |
| 67 | JB | | 38 | 1.83 | 606 | 332 | M |
| 68 | BB | | 35 | 1.98 | 1050 | 530 | F |
| 69 | TK | family history of heart attacks | 23 | 1.29 | 466 | 360 | M |
| 70 | VW | family history of heart disease, | 24 | 0.68 | 198 | 289 | F |
| | | depression, vascular disease | | | | | |
| 71 | JDN | | 49 | 0.76 | 446 | 587 | M |
| 72 | JF | | 42 | 2.09 | 395 | 189 | M |
| 73 | CWL | | 58 | 1.5 | 348 | 232 | M |
| 74 | RG | | 27 | 0.58 | 374 | 648 | M |
| 75 | RN | | 32 | 0.79 | 233 | 281 | M |
| 76 | LEL | | 47 | 0.43 | 99 | 230 | F |
| 77 | JCZ | heart disease, aneurysm history | 48 | 0.19 | 50 | 263 | M |
| 78 | MAC | | 49 | 1.41 | 255 | 181 | M |
| 79 | BPB | | 33 | 1.26 | 237 | 188 | M |
| 80 | CBT | | 22 | 0.76 | 142 | 187 | F |
| 81 | JB | | 22 | 1.36 | 370 | 272 | M |
| 82 | JST | high blood pressure | 50 | 1.74 | 327 | 188 | M |
| 83 | GRT | aneurysm history | 52 | 1.19 | 263 | 221 | F |
| 84 | JEF | smokes, cardiac disease history | 50 | 0.5 | 312 | 623 | F |
| 85 | JBB | | 48 | 0.33 | 82 | 247 | F |
| 86 | KR | | 29 | 1.72 | 873 | 508 | F |
| 87 | RR | | 25 | 2.06 | 952 | 462 | F |
| 88 | SK | | 42 | 1.5 | 437 | 291 | M |
| 89 | CB | smokes | 46 | 0.65 | 163 | 251 | F |
| 90 | PJR | | 53 | 2.17 | 779 | 359 | M |
| 91 | HT | | 44 | 1.84 | 479 | 260 | M |
| 92 | MT | | 33 | 0.38 | 394 | 1045 | M |
| 93 | CTH | | 60 | 1.16 | 169 | 146 | M |
| 94 | TAK | high cholesterol | 47 | 0.89 | 205 | 230 | F |
| 95 | CCK | | 47 | 1.35 | 289 | 214 | M |
| 96 | KJR | smokes and heart disease in family | 23 | 2.61 | 1404 | 538 | F |
| 97 | NS | | 26 | 1.82 | 522 | 287 | M |
| 98 | CFJ | | 44 | 2.57 | 360 | 140 | F |
| 99 | DB | | 30 | 2.02 | 562 | 278 | M |
| 100 | MR | | 59 | 1.55 | 343 | 221 | M |
| 101 | AL | | 24 | 1.34 | 693 | 517 | F |
| 102 | PB | | 53 | 1.72 | 368 | 214 | M |
| 103 | SS | | 46 | 0.38 | 96 | 253 | M |
| 104 | VRV | | 46 | 0.46 | 115 | 250 | M |
| 105 | THS | | 50 | 1.02 | 340 | 333 | M |
| 106 | ZC | | 44 | 2.02 | 394 | 195 | M |
| 107 | SJ | high cholesterol | 47 | 1.48 | 488 | 330 | F |
| 108 | MKP | | 63 | 1 | 164 | 164 | M |
| 109 | HLG | | 46 | 2.23 | 415 | 186 | M |
| 110 | KM | | 44 | 2.58 | 558 | 216 | F |
| 111 | SKO | cancer | 57 | 0.34 | 154 | 450 | F |
| 112 | EOM | | 46 | 1.01 | 734 | 649 | F |
| 113 | TK | | 34 | 1.85 | 1471 | 793 | F |
| 114 | DMH | | 43 | 2.01 | 1188 | 591 | F |
| 115 | LVMR | | 55 | 1.51 | 334 | 221 | M |
| 116 | CD | smokes | 34 | 0.99 | 223 | 225 | F |
| 117 | JTC | | 31 | 1.82 | 566 | 311 | M |
| 118 | RJC | pregnant | 31 | 0.6 | 146 | 244 | F |
| 119 | AP | | 27 | 1.64 | 394 | 240 | M |
| 120 | KL | | 26 | 1.61 | 411 | 255 | F |
| 121 | SKW | | 55 | 1.75 | 450 | 257 | F |
| 122 | JS | | 61 | 0.52 | 135 | 259 | F |
| 123 | DS | diabetes in family | 62 | 1.46 | 438 | 300 | M |
| 124 | RJ | heart disease in family | 48 | 1.04 | 276 | 265 | M |
| 125 | ESR | smokes and cancer in family | 28 | 1.34 | 224 | 167 | M |
| 125 | count | | ave | 1.36 | 395 | 291 | 122 |
| | | | stdev | 0.59 | 253 | 124 | |
| | | | | | min | 65 | |
| | | | | | max | 649 | |

| | |
|---|---|
| ave + 3 Stdev | 663 |

### Example 13

### Assay Validation Summary

The ELISA assay outlined in Examples 3-5 above has been validated by the tests outlined in Examples 6-14 above. The overall results data summary is listed in Table 12 below. As shown in Table 12, intra assay precision, inter assay precision, inter lot precision, linearity/range, recovery as well as matrix testing based on standard testing procedures have all passed the required acceptance criteria. The results obtained for the RIA and ELISA comparison testing were deemed acceptable since they were consistent with what was reported in the literature and the correlation between the two test methods was good with r=0.986. The interference study is not relevant as the current test procedure is not aimed to differentiate between desmosine and isodesmosine. The ELISA assay disclosed herein therefore is shown to be validated based on current standard measurement procedures in the field.

TABLE 12

| Test | Criteria | Results | Pass/Fail |
|---|---|---|---|
| Intra assay precision | CV ≦ 20% | ave CV = 19.44% | Pass |
| Inter assay precision | CV ≦ 25% | ave CV = 21.16% | Pass |
| Inter lot precision | CV ≦ 25% | oper 1 = 14.1% CV; | Pass |
| | | oper 2 = 15.2% CV; | |
| | | oper 3 = 7.9% CV | |
| Linearity/range | Desmosine moieties over range of | r = 0.995 | Pass |
| | use must fit standard curve with | r = 0.999 | |
| | an r ≧ 0.95. | r = 0.998 | |
| Recovery | Desmosine moiety recovery must | ave = 101% | Pass |
| | be ≧85% and ≦115%. | | |
| Comparison testing | In the comparison of ELISA with | 70% (ELISA reads higher | Pass |
| | RIA, a reference method for the | than RIA) but r = 0.986 | |
| | detection of desmosine, the | | |
| | results must correlate. | | |
| Interference | Difference ≦25% of control w/o | >100% difference for | Pass since |
| testing/specificity | interferent (i.e. desmosine moiety | IDES only | will measure |
| | specificity ≧75%) | | total DES/IDES |
| Matrix testing | Desmosine over range of use | r = 0.991 urine | Pass |
| | must fit standard curve with an r ≧ | r = 0.994 in buffer | |
| | 0.95 with no residual effect in | | |
| | human urine. | | |
| Reference Interval | Determine desmosine reference | 65-649 pmoles | NA |
| | interval in healthy subjects and | DES/mg C | |
| | include reference interval in the | | |
| | device labeling. | | |

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein.

1. A method for performing an Enzyme-Linked Immunosorbent Assay (ELISA) for quantitative determination of desmosine levels in a non-hydrolyzed urine sample of a mammal, the method comprising:
incubating the non-hydrolyzed urine sample with an anti-desmosine antibody in a well with an immobilized desmosine-capture conjugate to form a competitive combination for a first incubation time of no more than about 6 hours, wherein the antibody has appropriate affinity for both soluble desmosine and for the immobilized desmosine-capture conjugate wherein the assay volume is from about 1.3 to about 4 times the urine volume;
washing the well to remove anti-desmosine antibody not bound to the desmosine-capture conjugate;
adding an enzyme conjugated anti-antibody to the well to form a detection enabled sample well that is incubated for a second incubation time;
washing the well to remove unbound enzyme conjugated anti-antibody;
developing the detection enabled sample well by incubating an enzyme substrate with the detection enabled sample well for a third incubation time to form a detectable product;
detecting the amount of detectable product; and
estimating the amount of sample desmosine based on a comparison of the detected amount of detectable product with a standard curve.

2. The method of claim 1 wherein the anti-desmosine antibody was raised using a conjugate to a protein bound to desmosine with a protein crosslinking agent and the bound desmosine is conjugated with a macromolecule different from the protein used to generate the antibody and with a difunctional linker to form the conjugate.

3. The method of claim 1 wherein the second incubation time is no more than about 2 hours.

4. The method of claim 1 wherein the third incubation time is from about 3 min to about 20 min.

5. The method of claim 1 wherein the assay volume is from about 1.3 to about 4 times the urine volume.

6. The method of claim 1 wherein the desmosine-capture conjugate comprises desmosine conjugated with ovalbumin.

7. The method of claim 1 wherein the enzyme is a peroxidase and the enzyme substrate comprises tetramethylbenzidine.

8. A method for implementing a commercial ELISA assay for desmosine in urine, the method comprising distributing reactants to perform the method of claim 1.

9. The method of claim 7 wherein a kit is distributed comprising:
a high titer polyclonal anti-desmosine antibody raised with desmosine bound to a protein using a multifunctional protein crosslinking agent;
desmosine-capture conjugate comprising desmosine bound to a capture macromolecule through a linker molecule; and
wherein the desmosine-capture conjugate is effective for capturing in a prepared well to effectively segregate the desmosine-capture conjugate from soluble sample desmosine and wherein the desmosine-capture conjugate is effective to compete with un-bound desmosine for the anti-desmosine antibody and wherein the high titer polyclonal anti-desmosine antibody quantitatively captures un-bound desmosine from non-hydrolyzed urine with no more than a 6 hour incubation time.

10. A method for diagnosing aneurysm in a patient using an Enzyme-Linked Immunosorbent Assay (ELISA), the method comprising,
detecting the level of desmosine in the urine sample using the ELISA of claim 1; and
comparing the detected level of desmosine against a reference level of desmosine, wherein the reference level has been selected through the ELISA measurement of the level of desmosine detected from urine sample of healthy individuals to determine if a patient should be flagged as likely suffering from an aneurysm.

11. The method of claim 10 wherein at least 75% of adult aneurysm patients have a desmosine/creatinine concentration ratio greater than at least 75% of a healthy adult control population as measured with the ELISA assay.

12. The method of claim 1 further comprising:
generating a standard curve for an Enzyme-Linked Immunosorbent Assay (ELISA) for quantitative determination of desmosine in a urine sample of a patient, the generation of the standard curve comprising:
incubating an anti-desmosine antibody with a set of desmosine standard solutions each having a selected amount of desmosine spanning a desired range of desmosine concentrations in a separate wells having a selected amount of a desmosine-capture conjugate wherein desmosine-capture conjugate comprises a capture macromolecule, wherein the antibody has appropriate affinity for both the soluble desmosine and for the desmosine-capture conjugate and the well has an intermediate amount of desmosine-capture conjugate to provide a desired slope for a standard curve generated from the standard sample measurements over the range of concentrations corresponding to patient samples;
developing the incubated wells to illicit detectable signals from enzyme reaction products corresponding to the amount of desmosine in the standard; and
generating a standard curve based on the amount of signal measured as a function of desmosine concentrations in the standard samples.

13. The method of claim 12 wherein the ELISA measurement is performed through an ELISA kit.

14. The method of claim 12 wherein the anti-desmosine antibody is incubated for no more than about 6 hours.

15. The method of claim 12 further comprising, prior to developing the incubated well: washing the well to remove anti-desmosine antibody not bound to desmosine-capture conjugate; and adding a detection antibody conjugated with an enzyme wherein the detection antibody is bound quantitatively to the anti-desmosine antibody.

16. The method of claim 12 wherein the set of desmosine standard solutions comprises at least about 6 standard solutions.

17. The method of claim 12 wherein the set of desmosine standard solutions have concentrations within the range of about 1 picomole to about 32 picomoles.

18. The method of claim 1 wherein the anti-desmosine antibody has a dilution from about 1/3000 to 1/25,000.

19. The method of claim 1 wherein the anti-desmosine antibody has a dilution from about 1/3500 to 1/20,000.
